(19) United States
(12) Patent Application Publication
(10) Pub. No.: US 2026/0354911 A1
(43) Pub. Date: Nov. 12, 2026

(54) Robots Self-Directed by Quantum Computing and Classical Algorithms for Autonomous Physical Procedures in Medical, Dental, Cosmetic, Dermatology, Ophthalmology, and Veterinary Applications.

(71) Applicant: Keith Louis De Santo, St Petersburg, FL (US)

(72) Inventor: Keith Louis De Santo, St Petersburg, FL (US)

(21) Appl. No.: 19/532,226

(22) Filed: Feb. 6, 2026

Related U.S. Application Data
(60) Provisional application No. 63/772,905, filed on Mar. 17, 2025.
(60) Provisional application No. 63/788,022, filed on Apr. 13, 2025.
(60) Provisional application No. 63/814,532, filed on May 30, 2025.
(60) Provisional application No. 63/820,684, filed on Jun. 9, 2025.
(60) Provisional application No. 63/828,128, filed on Jun. 22, 2025.
(60) Provisional application No. 63/836,599, filed on Jul. 1, 2025.
(60) Provisional application No. 63/840,821, filed on Jul. 9, 2025.
(60) Provisional application No. 63/896,098, filed on Oct. 9, 2025.
(60) Provisional application No. 63/913,252, filed on Nov. 7, 2025.
(60) Provisional application No. 63/927,531, filed on Nov. 29, 2025.
(60) Provisional application No. 63/939,458, filed on Dec. 12, 2025.
(60) Provisional application No. 63/946,530, filed on Dec. 22, 2025.

(51) Int. Cl. A61B 34/30; A61B 34/10; G06N 10/20

(52) U.S. Cl. CPC A61B 34/30; A61B 34/10; G06N 10/20

(57) ABSTRACT

Robots self-directed by quantum computing and classical algorithms performing autonomous physical procedures in the medical, dental, cosmetic, ophthalmology, and veterinarian professions without guidance, or intervention by human operators. The system relies on comparisons of two data pools: one for real time patient data and the other archived patient data from prior patients. The comparisons are in image data, measurement data, and physical medical procedure data.

### PRIORITY APPLICATIONS

This application claims priority under 35 U.S.C. § 119(e) to the following U.S. Provisional Patent Applications:
63/959,324 filed Jan. 13, 2026, titled “Robots Self Directed By Quantum Computing and Classical Algorithms Performing Autonomous Physical Procedures in the Medical, Dental, Cosmetic, Ophthalmology, and Veterinarian Professions.”
63/946,530 filed Dec. 22, 2025, titled “Quantum and Classical Algorithms for Autonomous Physical Procedures in the Medical, Dental, Cosmetic, Ophthalmology, and Veterinarian Professions.”
63/939,458 filed Dec. 12, 2025, titled “Quantum and Classical Algorithms Directing Autonomous Physical Procedures and Applications in the Medical Profession.”
63/927,531 filed Nov. 29, 2025, titled “Quantum and Classical Algorithms for Autonomous Procedures.”
63/913,252 filed Nov. 9, 2025, titled “Automated medical, dental and veterinarian physical procedures and applications.”
63/896,098 filed Oct. 9, 2025, titled “Self Directed Autonomous Physical Applications with Processing Abilities.”
63/840,821 filed Jul. 10, 2025, titled “Automated Self Directed Applications.”
63/836,599 filed Jul. 1, 2025, titled “Self directed physical medical procedure autonomous MedsApp.”
63/828,128 filed Jun. 22, 2025, titled “Self Directed Autonomous Physical Medical Surgical Procedure.”
63/820,684 filed Jun. 9, 2025, titled “Self Directed Physical Medical Procedure Autonomous Applications.”
63/814,532 filed May 30, 2025, titled “Autonomous Testing of Matter using Robots, drones, Encased Areas, Sealed Doors, Stages and Jets.”
63/788,022 filed Apr. 13, 2025, titled “Algorithmic Medical Robotic Surgical Training and Learning Application.”
63/772,905 filed Mar. 17, 2025, titled “Algorithmic Medical Robotic Surgical Training and Learning Application.”

### FIELD OF THE INVENTION

The field of invention is the physical medical robotic invasive procedures and non-invasive medical procedures self-directed by computer software programs and algorithms programmed to provide step by step instructions to perform medical physical tasks autonomously. More particularly, the disclosure relates to systems and methods for training one or more models and for controlling one or more robots with mechanical arms, instruments, and sensors (hereafter referred to as robotics or robots) to autonomously perform invasive procedures and/or non-invasive applications in medical domains utilizing the trained data. In this embodiment, the procedures and applications are executed without human operator intervention, guidance, or monitoring. This field of invention is new with not much history. This new disclosure is autonomous physical procedures in the medical, dental, cosmetic, ophthalmology, and veterinarian professions (hereafter all referred to as physical medical procedures in the medical profession) without guidance, intervention, or monitoring by human operators or clinicians.

This new disclosure provides different methods of successfully completing an autonomous physical medical procedure utilizing robotics directed by algorithms without any human operator involvement. The methods are:
1—Step by step method called “Instructional Twin Procedures”, which are established instructional steps through training data before a procedure is started,
2—Bouncing of data comparison of real time patient data to archived data from many patients called “Bouncing”, and
3—Measured distances “Marked Positions” which are measured distances from areas “points or positions” on a patient “subject”, points on a surgical operating table, and 3 dimension points in the x, y, and z axes that are used for exact positioning of a body part, an organ or a robotic arm. The marked points use both marker devices and laser spot detection based on photodetectors. The methods involve training data in different manners. In an embodiment of this new disclosure, the instructional twin procedure method, the bouncing method, and the marked position method are used in single form or in combination with each other. Classical algorithms, quantum computing or a combination of both are used in this new disclosure called a hybrid algorithm.

This new disclosure has two embodiments, both separate systems but together form the entire system. First embodiment is the training system for data. Second embodiment is autonomous physical medical procedure utilizing the data from the first embodiment.

The first embodiment is trained manually by human operators or trained by the system itself or a combination of both manually and self-training called “MedsApp Training System” or “Training System”. The second embodiment called “Autonomous Physical Medical Procedures” or “Physical Medical Procedures” utilizes the data trained from the first embodiment to perform successful physical medical procedures without guidance, intervention, or monitoring by human operators or clinicians in medical professions. For purposes of this invention, autonomous will be a self-directed action or procedure without any input, guidance or monitoring from a human operator, group of human beings or a machine that is guided, managed, or controlled by a human operator. The autonomous action is controlled by computer software programs, algorithms, quantum computing programs and technologies or a combination of all three. Autonomous physical medical procedures will also mean “Self-Directed” physical medical procedures.

The first embodiment and the second embodiment form the entire system of the new disclosure which is named MedsApp.ai system. “MedsApp” or “System” and will be defined as the entire apparatus, its components, its operational systems, devices, tools, robotics, machines, algorithms, quantum technologies (qubits) and all its methods listed in this specification. Both embodiments utilize computer software programs, algorithms, quantum computational methods, machines, apparatuses, and devices that all perform autonomous physical medical procedures without guidance, intervention, or monitoring of human operators.

In an embodiment of this new disclosure, quantum computing technologies (quantum computing) are used to process data at a faster rate than classical algorithms using classical computer software devices which in turn means faster data processing for better precise performance.

### History of Medical Procedures and Training Of Doctors

History of medical procedures in the medical, dental, cosmetic, ophthalmology, and veterinarian professions with all afore mentioned professions practicing in the “medical industries”. Most of the current physical medical applications, physical procedures, and physical instructions are managed, guided, operated and given step by step instructions by doctors, their support staff and clinicians defined as “Human Operators” in the medical profession.

Formally in the medical professions, medical professionals and medical practitioners such as doctors were trained in a certain regimented way, graduated from medical school, received training from other doctors and go on to practice in the medical industry. Training manuals in book form, on the job training (sometimes by making mistakes) and medical professionals training by other medical professionals was the manner in which many doctors learned to perform medical physical surgical procedures. If a surgical procedure on a patient was successful and a doctor was responsible for the success, the question posed is—How did the doctor achieve their success in performing surgical procedures? The answer is—they were taught and trained to perform physical surgical procedures.

Most physical procedures, invasive surgeries, non-invasive physical procedures in the medical industry were performed by doctors either by their own hands or guiding, controlling, and utilizing mechanical arms of a machine. On some occasions, the doctors were assisted, replaced, or monitored by medical support staff and or clinicians with the medical procedures and applications. Guiding medical devices and robots with mechanical arms by way of the doctors hands (manual physical procedure) to perform physical surgical procedures was as far as autonomy was offered in the medical industry. Even with robots and training, medical professionals and their support staff are prone to making mistakes and errors before, during and after medical surgical procedures and associated physical applications such as taking the weight (non-invasive physical medical procedure) of a patient. The mistakes and errors the medical professionals and their support staff make include but are not limited to taking and writing incorrect notes, reading medical charts and X-rays incorrectly, leaving medicals tools and or prep materials inside a patient after surgeries, and not looking for the third choice if they only see two choices. This new disclosure eliminates many mistakes and errors in the medical professions. MedsApp algorithms and machine learning modules look far beyond the two choices with questions such as what if?

Present robotic applications in the medical industry and many others are either guided and or monitored by doctors, support staff or other skilled professionals hereafter known as “human Operators and Clinicians”. 100 percent autonomy does not exist. This type of human operator guided medical application still leads to human errors. The combining of robotics, mobile vehicles, mechanical arms, tools and devices with self-directed and self-learning algorithms with 100 percent autonomy created final outcomes that was once deemed beyond reach. For purposes of this new disclosure, all components, methods, and systems of software and hardware listed in this specification will be called “MedsApp.ai” or “MedsApp” or “System”. MedsApp automated machine learned medical procedures and applications through training of data improved overall outcomes through deep learning algorithms leading to enhanced successful physical surgical procedure outcomes through a faster and higher precision rate. MedsApp improved medical physical procedures and other types of non-invasive medical applications by automating and self-directing physical surgical procedures which eliminated human intervention whereby limiting human mistakes. The two arguments made for (using MedsApp.AI) and against (using human operators and clinicians) follows. Some widely stated opposition to the use of machines and their self-directed algorithms that replaces doctors are that patients will not want to talk to machines, and secondly, do not feel comfortable allowing machines to perform a self-directed physical medical procedure on them. The comfort factor must be present where they, the patient has become accustomed with person (doctor) to person (patient) interactions since they started visiting doctors at a young age. The argument for the MedsApp system utilizing machines and software systems is the simple fact that doctors and humans make mistakes, whereas the mistakes of the machines and their machine learning software algorithms become less and less after every physical medical procedure and application the machines and software perform and learn from. In order to act and think like a doctor, training of the system must be in the same physical manner as doctors were trained to perform surgeries, but with added data evaluation accelerators, surgically trained algorithms, tools, devices, and control units. In an embodiment of this new disclosure, self-directed machines through algorithmic process thinkers, extensive training through doctors, several robotic mechanical arms that feel pressure, with archived image and video data (incorrect data was used to gain correct data) instruct self-directed step by step procedures to perform in previous trained manners. After billions of procedures, images and video comparisons, MedsApp performed better than doctors by eliminating the wrong thought process, mistakes and errors doctors made in the past.

Robotic-assisted procedures with guidance and monitoring by humans have become increasingly common in human and animal healthcare. Existing systems typically rely on direct human operator supervised guidance with robotics having limited autonomy. These systems lack the ability to self-direct actions based on trained data through sensor fusion, predictive modeling, procedural state tracking, and adaptive real time decision making. Furthermore, current systems offer limited capability for cross disciplinary operation across medical, dental, cosmetic, ophthalmology and veterinary domains and professions. More importantly, supervised guidance by humans provides the propensity for human mistakes.

There exists a need for a fully integrated, algorithmically driven robotic control framework capable of autonomous execution of complex physical medical procedures. MedsApp system incorporates just that. It incorporates a trained system first, then continuous safety monitoring, multi modal sensing, procedural intelligence, and robust fail safe mechanisms that perform physical successful medical procedures without any human operator guidance therefore limiting human mistakes. The deployment of MedsApp brings precision to an apex, reduces patient physical trauma, and accelerates patient recovery, and decreases insurance costs and healthcare expenditures.

In an embodiment of this disclosure, MedsApp performs like a medical practitioner, doctor, or clinician but better without making many of the mistakes and errors medical practitioners and doctors have been prone to make in the past.

The idea of replacing medical practitioner/human operator with algorithms and machines when interacting with patients can be met with excitement, hope or distrust and dislike. The public research conducted by the inventor states either for or against with not many opinions that are neutral. The inventor has encountered both sides of the argument when researching public opinion on artificial intelligence (defined as the use of algorithms for this disclosure) “AI” and its medical applications. Some people, patients or groups of people find it awkward and impersonal to talk and interact with non-human devices impersonating humans. While others, find the interactions helpful since their biggest concern is getting the task at hand successfully completed with little or no negative effects to them. The choice is clear, be comfortable with a doctor who provides a sense of ease with personal interactions that may make an error or mistake in their treatments or physical actions. As opposed to human operators, have machines and algorithms that make decisions quickly, have access to a prodigious amount of successful physical medical procedure and application data, specific patient data, access to real time cell evaluation, and produce successful results. The real time MedsApp intraoperative image and video analysis, measured markers, and preset instructions that are able to be over ridden autonomously is a breakthrough in autonomous medical procedures. The argument against the new disclosure is simply some people, patients and groups of people will not embrace nor accept robots replacing doctors in the medical professions as well as other industries. This new disclosure may change their minds.

### BACKGROUND OF INVENTION

Robotic-assisted procedures are commonly performed with direct supervision and control by human operators and clinicians. Existing systems generally provide limited autonomy and typically rely on continuous human decision-making for planning, instrument manipulation, and response to intra-procedural events. Human operator-in-the-loop systems can also be susceptible to errors, mistakes, fatigue, and flawed execution. There remains a need for an integrated control framework that can (i) learn from large volumes of multi-modal procedural data, (ii) execute procedures using real-time sensing and closed-loop control, and (iii) apply robust self-reliant monitoring and fail-safe behavior to support autonomous operation.

As of the date of this new disclosure with inventor research commencing in 2016, robots and robotics have been performing surgical procedures on patients through the physical guidance and management of the robotics systems by doctors and their support staff further known as “human operators and clinicians” in this new disclosure.

With today's robotics being guided by human operators, many mistakes have been eliminated but there are still mistakes being made since the procedure is still being managed by human operators.

This new autonomous invention eliminates most human operator mistakes whereby after every successful training procedure or by autonomous procedure, the system learns and becomes more experienced at performing autonomous physical medical procedures which include surgical medical procedures (invasive) and testing medical applications (non-invasive) on patients.

This new disclosure trains data in three methods: manual training, self-training by the system or a combination of both. Any and all data whether or not partially successful or partially unsuccessful can be used for training purposes. A combination of both successful and unsuccessful training is where the beginning process of a physical procedure or application is successful but the end result is unsuccessful or vice versa. This new disclosure is trained by all data complied whereby small datapoints can be used such as images of a disintegrated gallbladder.

MedsApp manually trained data is developed in many different forms. The forms of data are manually trained data, self-trained data, and data from third parties. Some data from third parties include inputting data into the system by manually typing in data, requesting the system scan in hand written notes and convert them into electronic data for system archiving and storage, verbally speaking the data into the system via ambient technology, chatbot data that is electronically transferred from verbal conversations between robots and patients into system for system archiving and storage, or inputting digital scent data that can be manually transferred into system, utilizing human operators via computer software programs, algorithms, machines, devices, and apparatuses.

After the system has determined training is sufficient for certain physical medical procedures, which is a threshold of successful autonomous physical procedures on average of 400,000 times, the system determines which method of procedure is chosen by evaluating the data already obtained from patient. As mentioned, the 3 MedsApp methods are:

The first method is step by step procedure Instructional Twin Procedure that utilizes digital twin data from prior patients that is similar to the patient having the medical procedure performed on them by the MedsApp system hereafter called the “real time patient”. Similar data of the real time patient and similar data from archived patient data of pools are used in this method. MedsApp medical procedural data falls under one of two categories. The first is real time, in the now data pool from the patient having a physical medical procedure or physical medical application performed on them. The second data pool is the archived data from both training data and successful procedure data from former patients called archived data pools which is stored on computer software devices (or in qubits when using quantum computing). Similar data from both patient data pools is gender, age, height, weight and health condition of either, good, average or poor. Instructional Twin Procedure method also called “Twin” evaluates both data pools for which preprogrammed instructional steps will be taken in the Twin procedure. For example, decisions on Twin procedures are preprogrammed whereas a “game plan” is already laid out before the medical procedure starts.

The second method is a real time data comparison of both data pools (real time to archived) during the medical procedure called “Bouncing”. Bouncing is referred to data that is in constant comparison of real time patient data to archived data from many prior patients. This method is not pre-programed with instruction, but the system autonomously determines next steps with a high level of evaluation in real time. This method requires evaluation of a prodigious amount of data in very little time to make “on the spot” decisions. Quantum computing technologies enable not only quick evaluation but can evaluate real time data to archived data at a faster pace than classical algorithms. Classical algorithms can be used with this method when data pools are very similar with a standard deviation of 0.6 percent among in the now data pools of patient data to archived data. This method also utilizes the next method choice when challenges are encountered such as the removal medical procedure of a disintegrated body part such as a disintegrated gallbladder. Bouncing compares images of body parts, images of “moment in time” robotic movements, and images of where medical tools are during a medical procedure in small increments of time and space. For example, the constant comparing of data can guide the current real time procedure with past images, past positions and times as to where cutting of an organ was in relation to the x, y, and z axes and where each step in the medical procedure should end up with the compared images.

The third method is the marked positions with “Markers” method or “Measured” method. This method is unique whereby the method utilizes measured distances for exact placement of tools, devices and robotic arms from distances of patients body parts to markers on MedsApp medical surgical operating tables “MedsApp tables” or “tables”. The Measured method maintains many measured spaces to and from archived patients, MedsApp tables including all measured spaces around the two. Measured markers are also on robots, robotic arms, devices and tools. For example, MedsApp table has data transmission position devices “Makers” affixed to many areas on the table that transfer data in measurement form to all other measured spaces. In FIG. 8, FIG. 8.18, the positioned marker “Marker” transmits a marker signal that is measured to other markers on the table such as FIG. 8.14. That distance is calculated by mathematical equations converted into algorithms. As an example, that distance between 8.14 and marker 8.18 marker to is 6 feet, 1.25 inches. In FIG. 8.25, the marker affixed to a patient by a robotic arm, transmits a distance of 3.2 feet. Those distances of markers along with numerous others markers on tables, patients and further organs (levels of spaces), pinpoint exactly where body parts and organs are when also using the bouncing method 2 for image comparison. In an aspect of this disclosure, a laser spot detection system based on photodetectors is used based on a Gaussian spot model for markers on a subject for high accuracy. The technology is based on long wave infrared pulsed lasers. A grid using the cartesian plane is created by lasers that create accurate position spots on subjects which offers precise alignment of tools and devices to subjects body parts. In another aspect of this disclosure, quantum lasers also known as cascade lasers which use quantum mechanical principals to produce laser light at specific wavelengths to create 3 dimensional maps of a subject's body components for exact positioning of a marker.

In an embodiment of this disclosure, method 1, method 2, and method 3 (sections 0033, 0034, and 0035) can work in unison to perform an autonomous physical medical procedure without any intervention, guidance or input from human operators. The entire system is self-directed and makes its own decisions when data pools are trained and archived with enough data.

This new invention was trained to perform successful physical medical procedures on data from many different medical patients with many different images of external and internal patient body components such as organs, skin, bone, cells as well as other body parts such as disintegrated gallbladder pieces. All body components and pieces from humans and animals are hereafter referred to as “Body Parts”, “Body Components”, “Body Areas” or “Body Part Components”.

### SUMMARY OF INVENTION

In various embodiments, a system “MedsApp” includes two cooperating subsystems: (1) a training subsystem that obtains, curates, and stores training data and that trains one or more models; and (2) a self-directed procedure-execution subsystem that uses the trained data to generate and execute procedure plans and control commands for robots and instruments. The system maintains at least two data pools: a real-time data pool associated with a current subject (e.g., a patient) and an archived data pool comprising training data and data from prior subjects and prior procedures.

To autonomously perform a physical procedure, the system may apply one or more of the following procedure-control modes, alone or in combination:
Instructional Twin Procedure: A step-by-step procedure plan selected or generated prior to procedure initiation based on similarity between real-time data and archived data, and optionally executed with checkpoints and constraints.
Bouncing: A real-time comparison mode in which current sensor data is continuously compared against archived data to autonomously select next actions during the procedure.
Marked Positions: A measurement mode in which markers on a subject, operating table, instruments, and/or robots provide measured distances and reference geometry for precise positioning and trajectory validation.

In an aspect of this disclosure, the comparison of real time data to past archived data utilizes the speed of processing of data and calculations of quantum computing. Quantum computing enables MedsApp to compare large data pools of real time data such as specific body area images of a patient to archived data of past patient specific body area images for exactness of procedure and faster procedural operations. Also, archived distances to real time distances, and archived reference points to real time reference points through an expedited processing of all real time data to archived data enables faster processing while a patient on the MedsApp table.

In an aspect of this disclosure, MedsApp table reference points are measured to patient body component position points which are further measured in small increments of time to positions (cartesian x, y, and z axes) of robotic arms performing physical medical procedures for exact autonomous precision placement of an action. All of the data whether or not it is trained data or successful procedural data is data that enters data pools with little or no variation in data.

MedsApp manually trained data also includes human operator data obtained by robotic machines and their mechanical arms attached with tools and devices that are tracked, mimicked, measured or plotted in the x, y, and z axis (3 dimensional space calculations and imaging), video recorded, recorded with images, recorded by still image in space and time where the data is achieved in computer storage devices.

MedsApp training data is obtained, constructed, evaluated and stored by software programs, training algorithms, and in some embodiments by quantum computing. In this new disclosure, MedsApp was trained by human operators with the assistance of clinicians. Subsequently, the MedsApp system commenced the training of itself. The system when it encounters unknown data, procedures and or applications, it is designed to ask for help. The help is in 3 ways: it searches for answers through the internet, the databases it has access to including third party databases and at last resort will ask for human operator to help when all else fails. The shutdown of the system occurs when a challenge occurs that cannot be rectified after 400,000 loops of data or within 7 minutes time whichever comes first. An emergency alert is triggered to inform a bevy of human operators on call.

This new disclosure is a training system consisting of computer processing devices, data storage devices for computer processing devices, quantum technologies, software computer programs, and algorithms that direct machines, tools and devices to perform autonomous physical medical procedures in the medical, dental, cosmetic, ophthalmology, and veterinarian industries utilizing the trained data the MedsApp system creates.

MedsApp hardware and software components include every component listed in this specification. Some listed components work in combination with a few other listed components while other select listed components listed in this specification work with every and all other listed components listed.

This new disclosure employs computer software programs, algorithms, machines, sensors, tools, devices, safety mechanisms, quantum technologies, quantum computing qubit applications, cameras, image enlargement devices, video recorders, robotic touch pressure sensors, image perception through quantum computing comparative imaging, location and referencing points of patient body components, organ positioning markers, and specific body imaging (as to where organs should be and what shape they should be), normal cell imaging compared to abnormal cell imaging and evaluating and organizing next procedural steps, control units (for both electronic and hardware) for components, Universal Multipurpose Matter Detection Apparatus, medical devices, medical tools, lasers, lasers for 3D mapping, lasers for markers, 3 D mapping of the entire patient, 4 D (or more) mapping when utilizing quantum computing, (internal and external patient body maps for reference markers to spot any abnormality), 3 D printer, continual image cross checking for abnormal patient body components, patient body parts, patient body cells, all of which are operated, evaluated, given step by step instructions and self-directed by the system itself.

This new disclosure embodies a robotic system configured autonomously to perform acts traditionally performed only by human medical professionals and human operators, whereby the acts comprising: acquiring and recording patient information and clinical history; analyzing patient data to generate one or more diagnoses or differential diagnoses, selecting and planning one or more medical, dental, cosmetic, ophthalmology or veterinary procedures, positioning a patient and preparing a treatment site; selecting, configuring, and manipulating one or more instruments or tools; performing interventional acts on patient tissue, including cutting, drilling, ablating, suturing, stapling, injecting, replacing an organ, replacing a prosthetic limb or monitoring physiologic parameters and vital signs in real time; adjusting operating and surgical parameters, tool trajectories, and procedure steps based on sensed data, images, videos, and safety thresholds; detecting and responding to complications or adverse events; and generating, storing, and communicating procedure records, images, videos, and reports, and finally successfully completing a surgical procedure where the patient is better off than before—which is exactly what a doctor does.

The present invention relates to autonomous robotic systems, and more specifically to self-directed algorithmic control platforms enabling robots to perform medical, dental, dermatology, cosmetic, ophthalmology, and veterinary physical procedures with adaptive decision making, precision manipulation, integrated safety and self-monitoring subsystems without intervention from human operators. The new disclosure utilizes classical trained algorithms and quantum computer algorithms which also maintains self-learning capabilities. The Universal Multi-purpose Matter Detection Apparatus “UMMDA” is also a component to the system which detects abnormal cells in real time assisting the robots and drones and also self learns from both classical algorithms and quantum computer algorithms. In the medical patient room, during medical procedures and applications, a biosurfactant self-directed misting system is deployed (see FIG. 10), that provides the biosurfactant misting application through a table (FIG. 11) while 3D printers standby for the realtime construction of artificial body components “prosthesis” or prosthetic parts that the system requests or to contract to scale and replace broken body parts (see FIG. 13, FIG. 13.15).

Robots, machines, medical tools, devices and algorithms working together are first trained whereby after training, the system can successfully complete autonomous physical medical procedures and associated medical applications. Medical procedures utilize different types of mathematical equations converted into algorithms that provide step by step instructions each unique process in a set of procedures. Four main components (along with many others) are used in combination that without them, the new invention would not perform successfully. They are: 1—comparison of images and patient data between real time patients and archived patients, 2—quantum computing (see FIG. 5) for speed of bouncing (image comparison, measurement, and data comparison), quick calculations to fluctuating reference point devices for measurement on/in patients of body parts and MedsApp tables (see FIGS. 8), 3—tracking devices for medical tools, and medical devices for exact position to perform a physical procedure, reference points on surgical operating tables, mathematical measurements (see FIG. 9) to fill in unknown data if any and 4—a Universal Multipurpose Matter Detection Application (see FIG. 12, FIG. 12.26) for detection of abnormal cells.

In an aspect of this disclosure, MedsApp perform self-directed procedures in Medical, Dental, Dermatology, Cosmetic, Ophthalmology, and Veterinarian applications. The MedsApp system performs autonomous self-directed dermatology procedures. Dermatology is a branch of medicine that focuses on the diagnosis, treatment, and prevention of diseases and conditions affecting the skin, hair, nails, and mucous membranes. Dermatologists are specialized medical professionals trained to manage a wide range of skin-related issues. MedsApp and Ophthalmology applications. The MedsApp system performs autonomous self-directed ophthalmology procedures. Ophthalmology is a branch of medicine that focuses on the diagnosis, treatment, and prevention of diseases and disorders of the eye and visual system. It encompasses both medical and surgical care for various eye conditions. MedsApp and Dental applications. MedsApp system performs autonomous self-directed dental procedures. Dentistry is the science concerned with the diagnosis, prevention, and treatment of diseases of the teeth, gums, and related structures of the mouth and including the repair or replacement of defective teeth. MedsApp Cosmetic surgery or reconstructive surgeries. The MedsApp system performs autonomous self-directed cosmetic surgeries. Cosmetic surgery is a type of plastic surgery aimed at enhancing or altering a person's appearance for aesthetic reasons. It includes various procedures that can reshape features, smooth wrinkles, or lift sagging skin, often to boost self-confidence and self-esteem. MedsApp Veterinarian applications. The MedsApp system performs autonomous self-directed veterinarian applications. Veterinarian or pet doctor is a medical professional who is qualified and authorized to practice veterinary medicine, treating health conditions and injuries in animals and mammals. They also play roles in animal health management, reproduction, and preventive care. The MedsApp training applications provided exactness in many dental and medical procedures such as deploying a medical screw. In some MedsApp applications, screw positioning in bone fracture through MedsApp procedure in thoracolumbar fractures, found that the AI-robotic approach yielded a higher successful screw placement as opposed to screw misplacement, and an angle that was deemed to be unsuccessful which were both performed by human operators. The same MedsApp dental implant procedure leads to successful osseointegration or the infusing the dental implant with the surrounding bone of the patient. Comparative images of thousands of data points make this procedure successful.

This new disclosure and all of its hardware components, software components, systems, modules, algorithms, operations, applications and procedures listed in this specification will be known as “MedsApp” or “MedsApp AI” or simply “system”. The MedsApp system is designed to train data first, then apply that trained data for the administration of self-directed autonomous medical procedures and medical noninvasive applications without any intervention, guidance or monitoring by doctors and or medical staff professionals.

The MedsApp data training system and its methods employs both a manual system and a self-directed training system “self-training”.

The MedsApp system relates to systems and methods for training one or more models using training data, and for controlling a medical robotic system to autonomously perform one or more invasive procedures and/or non-invasive medical applications without requiring human operator/clinician intervention, guidance, or monitoring.

In an embodiment of this disclosure, the present disclosure comprises systems and methods including algorithms, software applications and robotic hardware configured to train one or more machine learning models using training data and to control one or more medical robotic instruments to perform self-directed invasive procedures and/or non-invasive medical applications in real time without requiring human operator/clinician intervention, guidance, or monitoring. In a further aspect of this new disclosure, large numbers of high resolution images and high resolution videos of past specific physical medical procedures are combined together for a successful one moment in time (can be 10,000th of a second) of a correct image and video that corresponds exactly to a real time patients imaging of a body area. Images combined with other separate images of different patients as well as videos combined with other separate different patient videos are also used. All images and videos can be used separately or in combinations of each other. Time of each procedure is also calculated for accuracy of each successful physical medical procedure from a past procedure compared to a present procedure.

MedsApp.AI system comprises an autonomous computing platform executing a suite of hybrid algorithms that direct robots, mechanical arms, electronic devices, mechanical devices, tools, sensors, medical devices, mobile vehicles, control actuators that generate procedural plans, step by step instructions with looped feedback on other procedural options and applications that can be implemented. MedsApp robotic devices through algorithms continually provides feedback (looped feedbacks) for constant evaluation of medical procedures, step by step instructions, application, and operations.

Summary of self-directing physical procedures and applications. MedsApp system trains data and implements the data trained into self-directed physical procedures and physical applications in the medical, dental, cosmetic, dermatology, ophthalmology and veterinarian industries without the intervention, guidance and monitoring from human operators and clinicians. The MedsApp apparatus, systems and operations are fully autonomous and provide a method for training and procedures of intake patient procedures.

### BRIEF DESCRIPTION OF THE FIGURES

The accompanying Figures illustrate non-limiting example embodiments. Reference numerals and figure labels (see reference Figure document) are provided for convenience and are not limiting.

FIG. 1—Patient intake algorithm.

FIG. 2—Training of gallbladder removal (example).

FIG. 3—Classical autonomous gallbladder removal algorithm (example).

FIG. 4—Quantum technology overview.

FIG. 5—Quantum-computer training algorithm.

FIG. 6—Flowchart of an autonomous self-directed quantum algorithm.

FIG. 7—Flowchart overview of a hybrid self-directed algorithm.

FIG. 8—Subject on a surgical operating table with markers and reference points.

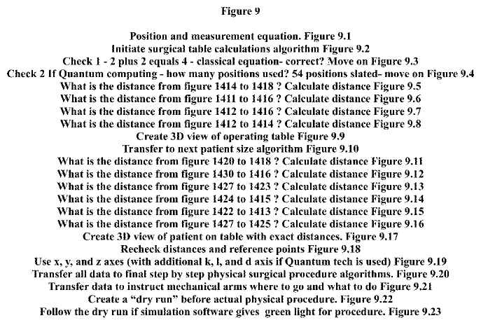

FIG. 9—Flowchart of measurement and reference-point calculations.

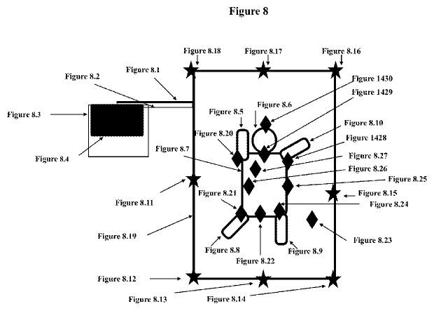

FIG. 10—Biosurfactant mixture tank (example).

FIG. 11—Biosurfactant mist flow through an adjustable operating table (example).

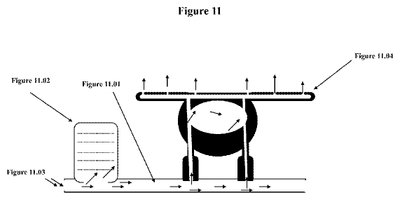

FIG. 12—Example procedure room layout.

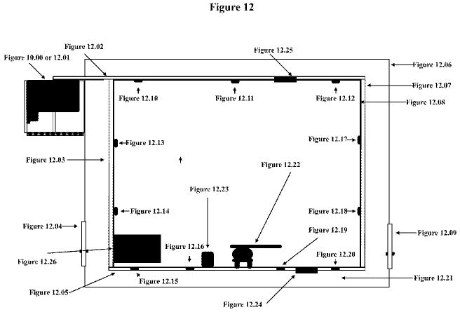

FIG. 13—Example operating room with robotic arms and optional drone assistance.

FIG. 14—Mechanical arm example (rotating elbow and extendable forearm).

FIG. 15—Attachment platform for tools/devices on a mechanical arm.

FIG. 16—Side view of a device/tool attachment platform.

FIG. 17—Tracking glove example.

FIG. 18—Forceps example (with optional tracking features).

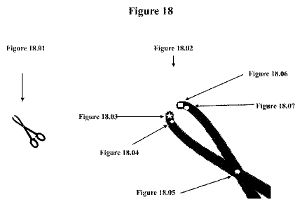

FIG. 19—Example stationary quantum computer.

### DETAILED DESCRIPTION

The following description is provided to enable one skilled in the art of automated physical medical procedures and training thereof to use the disclosed subject matter and to set forth the best mode contemplated. The disclosed embodiments are illustrative and may be modified without departing from the scope of the claims. The following sections [0054] to [0082] state definitions and show non limiting examples and components of the new disclosure.

### Definitions and Terminology

MedsApp.ai: The overall system including software, models, algorithms, robots, instruments, sensors, computing hardware (classical and/or quantum), data stores, and associated methods described herein.
Autonomous/Self-Directed: Operation in which procedure steps and control actions are generated and executed by the system without real-time human intervention, guidance, or monitoring. The system may have an aspect of this disclosure to be equipped with a component to escalate to search other third party data bases for answers to unknown challenges that are not indexed in archived data pools or seek a human operator under certain configured fail-safe conditions.
Real-Time Data Pool: Data obtained from a current subject during intake, historical health care data, monitoring, and/or procedure execution, including images, video, measurements, physiological signals, tool tracking, and matter-detection results.
Archived Data Pool: Data obtained from prior subjects including all their history and prior procedures and/or from training and simulation, stored for retrieval and comparison against real-time data.
Hybrid Algorithm: A combination of classical algorithms (bit-based processing) and quantum algorithms (qubit-based processing) used separately or together for modeling, optimization, decision-making, and autonomous physical medical procedures.
Instructional Twin Procedure: A preselected or generated step-by-step procedure plan derived from archived data and adapted for a current subject. In some embodiments, the plan is represented as a digital twin of procedural state and steps.
Bouncing: A real-time control mode in which current procedure data is repeatedly compared against archived data to determine next actions at a selected time granularity.
Marked Positions/Markers: Reference points (active or passive) disposed on a subject, an operating surface, instruments, and/or robots to provide measured distances and geometric constraints for positioning and trajectory validation.
Checkpoint Loop: A software mechanism that periodically saves procedure state and enables rollback or branching to alternate actions when thresholds are exceeded or errors are detected.
UMMDA: Universal Multi-Purpose Matter Detection Apparatus, configured to test matter samples (e.g., blood, urine, stool, sputum, tissue) and to output results usable by the control system, including detection of pathogens and/or abnormal cells.
Procedure Demand Score: A numeric score, e.g., on a 1-100 scale, computed from intake and diagnostic data to recommend monitoring, additional testing, or performance of a selected procedure.

### System Architecture

In an embodiment of this disclosure, MedsApp comprises an autonomous computing platform coupled to one or more robots, robotic manipulators, end effectors, sensors, and diagnostic devices in an operating environment. The computing platform can execute perception, localization, planning, control, and safety modules, and can access local and/or remote data stores containing the archived data pool. Communication among components can be wired and/or wireless and may include secure channels, including quantum-key-based schemes.

### Training Subsystem

The training subsystem obtains training data from one or more sources, including (i) recordings of human-performed or human-supervised procedures, (ii) robot-performed procedures, (iii) simulated procedures using simulation software, and (iv) third-party datasets. Training data can include multi-modal sensor streams such as images, video, depth maps, LiDAR point clouds, ultrasound/CBCT/MRI, force/torque and tactile readings, tool pose and trajectory traces, marker measurements, and audio (including ambient speech). The training subsystem stores labeled and/or unlabeled data in the archived data pool and trains one or more models for perception, diagnosis support, trajectory generation, and procedure-state tracking. Simulation software may be used for portions of training, including generation of procedural digital twins, synthetic sensor streams, and validation of control policies under varied anatomical geometries and constraints. Training can be performed manually by human operators, by self-training of the system, or by a combination thereof.

### Autonomous Patient Intake

Before initiating a procedure, MedsApp can autonomously intake a subject and create or update a subject record. Intake can include collecting demographic information, medical history, and environmental and lifestyle information; measuring vital signs; capturing images; and obtaining matter samples for testing. In some embodiments, matter samples including blood, urine, stool, sputum, and tissue are analyzed by UMMDA to detect pathogens, microbes, and/or abnormal cells in real time. The intake data is stored in the real-time data pool and may be appended to the archived data pool after completion of the encounter.

In some embodiments, the system computes a Procedure Demand Score (e.g., 1 to 100) based on one or more signals derived from intake data. A lower score can trigger monitoring and follow-up actions, while higher scores can trigger additional testing and/or selection of an autonomous procedure. For example, a score above a configurable threshold (e.g., 50.1) can trigger additional tests, and a higher score (e.g., 92.6) can trigger initiation of a selected procedure. The scoring can aggregate weighted evidence including symptoms obtained via ambient listening, imaging findings, and UMMDA test results.

### Data Pools, Indexing, and Comparative Control

The procedure-execution subsystem can access a real-time data pool associated with a current subject and an archived data pool comprising prior procedures and training records. In some embodiments, the system computes a difference metric between the two pools (an effect differential) and selects actions to reduce deviations from expected states. In some embodiments, comparisons are performed across at least images, measurements, tool trajectories, and end-state outcomes. Certain comparisons can require minimum dataset sizes (e.g., at least 40,000 data points for a selected feature class) to support statistical confidence.

In some embodiments, the system forms “baskets” or codes of data comprising grouped features (e.g., images, measurements, marker coordinates, tool trajectories, physiological signals) associated with a procedure step or state. These baskets can be indexed for retrieval and used as step definitions for the Instructional Twin Procedure and for real-time comparisons in Bouncing mode. For positioning and motion, the system may represent the subject and instruments in a Cartesian reference frame (x, y, z) and may track additional degrees of freedom including orientation (roll, pitch, yaw). In some embodiments, the system may represent uncertainty or alternative hypotheses over states and trajectories, including via probabilistic and/or quantum technology representations.

### Autonomous Procedure-Control Modes

### Instructional Twin Procedure

In an Instructional Twin Procedure mode, the system selects one or more archived cases that are similar to the real-time subject data (e.g., by age, anatomy, measurements, condition indicators, and imaging features). The system generates or selects a step-by-step plan prior to procedure initiation, including tool selection, approach parameters, and expected state transitions. During execution, the plan can be validated at checkpoints and updated when observed data deviates from expected states.

### Bouncing Mode

In Bouncing mode, the system performs repeated real-time comparisons between current sensor data and archived data to autonomously select subsequent actions during the procedure. Comparisons can be performed at selected time increments, including fine-grained increments (e.g., 1/10 second or finer such as 1/10,000 second) for tool movement and tissue-state evaluation. In some embodiments, quantum computing resources are used to accelerate large-scale comparisons and optimization. In some embodiments, classical computing alone may be used when the real-time data pool and archived data pool are sufficiently similar, such as when a standard deviation between comparable feature sets is below a threshold (e.g., 0.6 percent).

### Marked Positions Mode

In Marked Positions mode, the system uses markers disposed on the subject, operating surface, robots, instruments, and/or room reference points to obtain measured distances and reference geometry for positioning and trajectory validation. Markers can transmit signals (e.g., electromagnetic, optical, laser, or wireless) and can be used to triangulate positions in a Cartesian coordinate system. As a non-limiting example, a first marker on an operating table may be measured relative to a second marker at a known distance (e.g., 6 feet and 1.25 inches), and a marker affixed to a subject may be measured relative to the table markers (e.g., 3.2 feet) to maintain a consistent reference frame throughout a procedure. Marked measurement data is also used on and in specific organs, body parts and or position of bodily fluids. In some embodiments, the system uses two or all three modes in combination. For example, an Instructional Twin Procedure plan can provide baseline steps, Bouncing mode can provide real-time branching decisions, and Marked Positions can provide geometric constraints for tool placement and motion verification.

### Computing Hardware

The computing platform can include one or more processing devices and memory. Example processing devices include general-purpose CPUs, parallel processors such as GPUs, machine-learning accelerators such as TPUs and NPUs, DSPs for sensor streams, reconfigurable logic such as FPGAs, application-specific integrated circuits (ASICs), microcontrollers (MCUs), and system-on-chip (SoC) devices. In some embodiments, the platform further includes one or more quantum processing units (QPUs) used for quantum algorithms, optimization, and/or accelerated comparison against archived data. Example memory and storage devices include volatile memory (e.g., SRAM and DRAM), non-volatile memory (e.g., flash/NAND, EEPROM, MRAM, ReRAM), and persistent storage systems including local drives and networked and cloud storage. Data stores can include relational databases, NoSQL databases, object storage, logs/queues, and version-controlled repositories. In some embodiments, distributed ledgers (e.g., blockchain) may be used for selected audit and integrity functions.

### Robots, Instruments, Sensors, and Tracking

MedsApp can include one or more stationary or mobile robots and one or more robotic manipulators with multiple degrees of freedom. End effectors can be interchangeable and can include cutting, grasping, suturing, drilling, polishing, injection, and ablation tools. Sensors can include cameras, depth and time-of-flight sensors, structured light devices, LiDAR, radar, ultrasound, CBCT, MRI, force and torque sensors, tactile sensors, acoustic or ultrasonic transducers, thermal sensors, and physiological monitors. Tracking can be performed using markers or tracking objects affixed to instruments, gloves, garments, robots, or subject anatomy, including reflective or brightly colored fiducials and embedded electromagnetic micro-sensors. Tracking signals can be wired or wireless and can include optical, electromagnetic, inertial, ultrasonic, and/or radio-frequency signals. In some embodiments, tracking devices are embedded in instruments or disposed on the surfaces of instruments and tools. In some embodiments, tracking gloves or other wearable tracking devices are used during training to capture fine-grained hand and finger motion, and the captured motion is mapped to robotic manipulators for imitation learning and trajectory generation. For 3D spatial perception, the system can employ multi-modal sensor arrays including cameras with depth sensing, LiDAR, and radar to build point clouds and meshes of the operating environment and subject anatomy. Example algorithms include simultaneous localization and mapping (SLAM), sensor fusion, and object detection and tracking in 3D. Green screens or background reference surfaces may be used during training or execution to improve segmentation and tracking quality. LiDAR and laser-based measurement can be used to project patterns onto anatomy, instruments, and robot structures, and to compute distance and pose from pulse timing or phase information. In some embodiments, CAD models and/or shot plates are used for alignment and validation. In some embodiments, the system includes digital scent technology configured to detect odors in an operating environment and categorize odors for archiving and correlation with procedure states and/or diagnostic outcomes.

### UMMDA and Matter Detection

UMMDA can be configured to receive matter samples via a receptacle and to test samples using one or more analytical modalities. Non-limiting examples include detection of pathogens and microbes, analysis of biomarkers, and classification of normal versus abnormal cells. UMMDA outputs can be used during intake and during procedures to guide decision-making and to trigger actions such as additional sampling, procedure selection, or environmental mitigation. In some embodiments, matter evaluation can include microscopic imaging (e.g., electron microscopy) and image-enlargement processing. In some embodiments, image enlargement can be performed at very high magnification, and quantum computing resources may be applied to accelerate image enhancement and classification. In an aspect of this disclosure, autonomous application utilizing robotics for obtaining a matter sample from a subject further placing that sample on a stage (closed area) of an electron microscope to be evaluated and archived in data store.

### Biosurfactant Misting and 3D Printing

In some embodiments, the procedure environment includes a biosurfactant misting system comprising a mixture tank and delivery channels integrated with an operating table. The system can autonomously trigger misting based on UMMDA results or other sensors to reduce microbial load before, during, or after procedures. In some embodiments, one or more 3D printers are positioned within or near the procedure environment and may be maintained in a standby state. The system can request fabrication of replacement components such as prosthetic parts or patient-specific fixtures, and robots and/or drones can assist with printer operation, retrieval, and staging of printed components.

### Mobile Platforms and Remote Operation

In some embodiments, MedsApp includes or interfaces with a mobile platform such as an autonomous vehicle configured to travel to a subject location to perform intake, monitoring, and/or selected applications. As a non-limiting example, an autonomous “MedsApp bus” may be deployed for remote checkups or follow-up testing. Mobile platforms can include ground, air, or water vehicles and can optionally carry robotic manipulators and sensor suites.

### Internal Roaming Robot for Training and Inspection

In some embodiments, MedsApp includes a miniature internal roaming robot configured for operation inside a cadaver or anatomical model during training. The roaming autonomous robot can include a camera or other sensors and can traverse within a body cavity to capture internal imagery and measurements that are stored in the archived data pool.

### Safety, Checkpoints, and Fail-Safe Behavior

MedsApp can include a safety module configured to evaluate operational risk using force thresholds, proximity to sensitive anatomy, physiological sensor data, deviations from expected procedural state, and other safety constraints. When thresholds are exceeded, the system can autonomously halt, revert to a safe pose, roll back to a checkpoint, branch to an alternate plan, and/or request human operator assistance. In some embodiments, if a challenge cannot be rectified after a predetermined number of evaluation loops (e.g., 400,000 loops) or within a predetermined time window (e.g., 7 minutes), the system triggers an emergency alert to notify on-call personnel (Human Operators) and transitions to a safe state. A manual power-disconnect breaker can also be provided. Checkpoint loops can be implemented such that procedure state is periodically saved (e.g., tool pose, planned trajectory, observed anatomy classification, and recent sensor history), allowing the system to resume from the last safe state or branch to an alternative action sequence when a deviation is detected.

### Quantum Computing and Quantum Technologies

In some embodiments, MedsApp uses quantum computing resources to accelerate selected computations, including large-scale data comparison, optimization, pattern matching, and model training. Quantum computing can use qubits and quantum algorithms and may be implemented using superconducting qubit chips and/or other quantum modalities such as trapped ion systems, photonic quantum computing, neutral atom systems, and spin-based qubits in semiconductors. Example quantum hardware components can include Josephson junction circuits and transmon technologies, along with control stacks for qubit manipulation and readout. Quantum resources can be local or remotely accessed (e.g., cloud-based quantum computing). In some embodiments, secure communication among components can use quantum key distribution. In some embodiments, quantum-enhanced sensing and imaging may be employed, including quantum lasers and quantum-enhanced tomography, to improve spatial resolution and tissue characterization.

### Example Procedures and Applications

Non-limiting examples of autonomous procedures and applications include biopsies, organ removal procedures (e.g., gallbladder removal), dental implant procedures, dermatology screening and treatment, ophthalmology procedures, and veterinary procedures. In each example, the system may combine intake testing, multi-modal sensing, comparative control using the data pools, and safety monitoring to execute procedure steps and document outcomes. As one illustrative example, intake data can indicate a condition consistent with gallbladder pathology based on a combination of symptoms, imaging, and matter testing. The system can compute a Procedure Demand Score and, when a threshold is met, can initiate an autonomous gallbladder procedure plan using the archived data pool. During execution, the system can use multiple cameras and tracking measurements, continuously compare real-time observations to archived cases, and apply biosurfactant and other environmental controls as needed, while enforcing safety thresholds and fail-safe behavior.

As of the date of the first priority document of Mar. 17, 2025, physical medical procedures utilizing quantum computing or classical algorithms for either invasive or non-invasive medical procedures from start to finish of the entire process guided by 100% computer software programs and algorithms has not been done.

This new disclosure listed in its entirety in this specification, is an autonomous method, an autonomous application with combinations of components that provide step by step physical medical procedures without an external source that intervenes in the process other than the computer software programs and algorithms that direct the process, steps and its procedures. This new disclosure utilizes computer software programs, algorithms, and machines for every facet of intake of a medical patient, any patient intake physical application, any physical procedure upon a subject, or all encompassing of data gathering for a subject whereby all facets are autonomous where the system directs itself in all aspects.

In an aspect of this new disclosure, the MedsApp.ai system with all its components listed in this specification is autonomous and performs physical medical procedures and non-invasive medical applications without any guidance, intervention, or monitoring from human operators. Both combined software and hardware technologies, machines, tools and devices utilize many types of software languages such as python, qubits (quantum computing) or older computer software languages such cobol, and simulation software. MedsApp hardware components consist of UMMDA, chemical testing devices, DNA testing devices, robots, drones, watercraft, watercraft for internal body roaming vehicles (with robots, drones, watercraft and internal body roaming vehicles being tethered or untethered), electronic devices, mechanical devices, sensors, markers, tracking devices (gloves/clothes), ambient listening technologies, chatbots, robotic arms (can rotate 360 degrees), medical tools/devices, headset googles, green screens, and quantum computers.

In an aspect of this new disclosure, data is trained manually by human operators through their efforts or by human operators guiding robotics. Human operators can input data into the system by typing the data into the system, speaking the data verbally into the system via ambient listening technology or by the tracking and mimicking of robotic movements and measurements allowing the system to track, learn and be trained from robotics operated and guided by human operators. MedsApp system operates autonomously by combinations of machines, apparatuses, tools, devices which are directed by computer software programs, and algorithms after appropriate training is completed for specific medical procedures.

In an embodiment of this disclosure, comparative imaging, reference points, exact measurement through reference points and algorithms through mathematical equations perform physical medical procedures flawlessly after training utilizing a suite of algorithms. This new invention compares images of normal patient body parts to abnormal body parts using classical algorithms utilizing contemporary computer software devices and quantum computing devices utilizing quantum technologies and qubits. Quantum computing enables the new invention to quickly compare many different data points including images in a short amount of time when compared to classical algorithms.

Training the MedsApp system. The system cannot perform procedures and applications without trained data. The system is trained through both successful and unsuccessful procedures, comparative image from successful images to unsuccessful images, and past physical training of successful medical applications. Then MedsApp completes the assigned tasks, procedures and final goals through the trained data.

Each and every MedsApp particular procedure and application must be trained for that specific physical medical procedure or specific physical medical application. MedsApp system knows itself if it is ready for procedures on patients (fully autonomous with no oversight from human operators) in real time applications and or procedures by performing loops through modules for each and every single procedure and or application it embarks upon. An example is for an application of weight calculation of a patient that MedsApp application did not need as much training as an invasive procedure such as an MedsApp organ or body part removal.

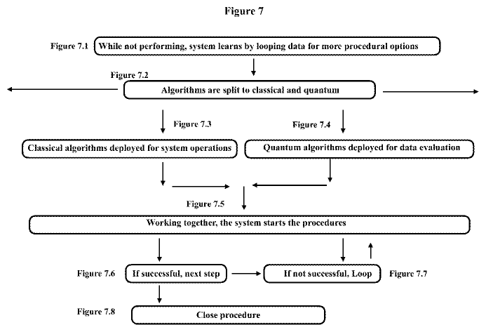

The MedsApp hybrid Algorithms. This new algorithmic invention training operation is a combination of training algorithms and machines that removed monitoring, guidance and intervention from many different industries from human operator management. This new invention of both software and hardware MedsApp components archives unsuccessful and successful medical procedure and application results, learns from both and forecasted events in individuals, groups as well as geographical areas in many industries. See FIG. 7 for an overview of a flowchart for a hybrid algorithm.

The invention has two main goals to: (1). Have medical professionals train machines and their software applications to teach, archive and build databases on which medical procedures and applications were successful, which were not successful, which procedures could be improved upon, and what data and information is not known as of yet. The MedsApp compares the data obtained to databases from many doctors and patent data worldwide to keep track of what is developing in the world of procedures, applications, sicknesses and disease. As the system evolves with more data obtained by the system that it learns from, the expertise will be from thousands of doctors not just the few whom trained the system as well as data from thousands of patient procedures worldwide. The algorithms will spot patterns from the multiple data points and also forecast events in the medical professions. Misdiagnoses will become easier to spot and in time many will be eliminated with the system trained to forecast if a misdiagnosis made be made. The MedsApp learns and forecasts medical events from the medical surgical procedures listed in this specification. (2) To rely less and less on medical professionals for training, monitoring and intervention for every and all physical medical procedures and medical applications. The system will rely more and more on the autonomous MedsApp machine and software, algorithmic applications for training itself. MedsApp self-training is when the system trains itself without any intervention from human operators. The system utilizes data it already has and also obtains the data by its own actions and trains itself from that data without any input from external sources. In an aspect of this new disclosure, both MedsApp manual training data and MedsApp self-training data are both used to train the MedsApp system. At all times of MedsApp autonomous operation, training still takes place to learn more about every procedure, application or system operation. Most of the MedsApp training will be self-training where some manual training will occur in later stages of each trained application. This manual training occurs long after the MedsApp has completed autonomous self-directed procedures when information encountered by the MedsApp system is unknown. The overall objective is to learn from real time procedures during any and all surgical procedures including but not limited to the detection and treatment (if possible) of abnormal cells, detection of abnormal organ function, detection of abnormal bone structure and possible real time action treating such issue, hair, bone, dictation of abnormal bodily fluids and apply that gained knowledge in the final autonomous goal of a successful procedure and or application. The MedsApp system will learn in the future about every aspect of a human, animal and plant which will also include psychological traits and also learn from those behaviors.

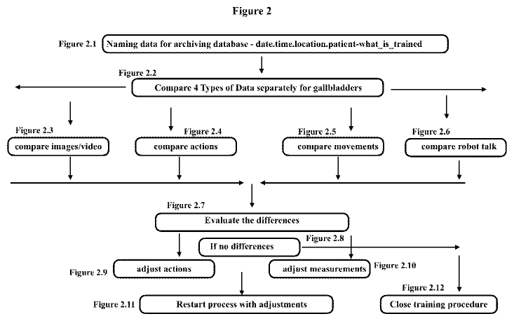

This new disclosure is a physical medical procedure machine learning system that learned from training software algorithms by intersecting surgical medical procedures and their corresponding medical applications performed by human operators and robots. The system is now able to train itself after most of the training was via a combination of human operators directing robots to perform medical procedures. The new training application combines algorithms, hardware, and simulation software programs to train itself for all physical medical procedures and applications. The system learned each time a specific or different type of surgical medical procedure complied data by the intersection of human operators, simulation software, simulation hardware, machines, tools, devices, and algorithms. The final goal of the MedsApp system is to perform all particular trained physical medical procedures and applications autonomously and self-directed by algorithms that were once trained by human operators, in conjunction with robots and robotics. The first self-directed medical procedures that were trained and performed with human operator assistance was biopsies and gallbladder removals. A classical algorithm overview is in FIG. 2.

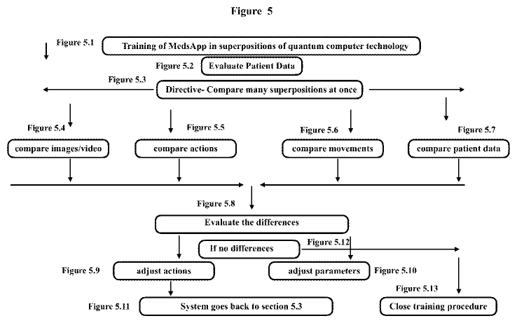

In an embodiment of this disclosure, there are two suites of algorithms of the MedsApp: (1) The training of the system using human operators and self-training (a total of two training modules) and, (2) the autonomous self-directed successful medical procedures utilizing the trained data of the system (a final medical procedure with many methods). The two main modules, systems and operations both utilize a hybrid algorithmic application. FIG. 5 is an overview of a quantum computing training algorithm.

This new disclosure provides an autonomous robotic system powered by self-directed suite of algorithms that manage and direct all aspects of training the system to further autonomous application. The system was trained first and continues to gain training through procedures successfully and unsuccessfully completed on patients, cadavers, and simulation software. The new MedsApp system collectively improves physical medical procedure effectiveness by continued training. The system is able to deploy an autonomous robotic procedure and application (with no human intervention) to complete self-directed tasks with precision that led to better patient outcomes, significant reduction in complication rates across medical, dental, cosmetic, dermatology, ophthalmology and veterinarian professions hereafter all together referred to as medical.

The combination of skilled medical doctors combined with robots (and sometimes drones and simulation software programs) for training purposes perform physical medical procedures on human beings and animals for training the MedsApp that are electronically archived in storage devices through the implementation of several components, tools, devices, and algorithms. MedsApp ambient listening and talking technology also guides the MedsApp with both the training and the physical medical procedures. Ambient listening and talking technology can guide the AI MedsApp with training, patient intake, training by a human being (most likely medical clinicians) with their verbal ideas, movements, and verbal guidance when training. The information from the human operator training the MedsApp speaks slowly so the ambient voice to text is transferred into the electronic training data. The ambient talking technology may also guide the MedsApp with performing physical medical procedures. There are many more methods, applications and procedures that the MedsApp ambient listening technology assists with include checklists before, during and after an application or procedure. A robot in the MedsApp system combines verbal details of a procedure to the exact movements of itself which are tracked and mimicked by algorithms. The data garnered during the training procedure is verbally, mimicked in movement and measurement through video and images, and physically plotted (dot plots through mathematical derivatives) in space using x, y, and z axis. A reference point for the cartesian plane may be a point on the wall of the MedsApp room. MedsApp system combines many different training components, methods which include verbal training. MedsApp robots during training procedures speak and detail all actions through verbal wording in english (as well as other languages) which in turn is also combined with physical actions and movements which is further converted in electronic data, 3D images and video as well as mathematical equations.

MedsApp Software Types. A plethora of computer software programs with different computer software languages complete many tasks that can be purchased from third parties. Some of these algorithmic codes come with support to revise the application to complete certain tasks. Through ease of purchase and ready out of the box software code, autonomous machines controlled by algorithms can complete physical chores. One such task was to perform an oxygen test of a patient by using software not compatible (unable to communicate) with the MedsApp system but was revised to do so. This new disclosure has achieved success with automation and self-directed medical procedures. In an embodiment of this new disclosure, algorithms and computer software programs are written in computer software languages that self-direct all operations of the MedsApp system “system”. Once a power source is supplied to the system, and the system is turned on, it is autonomous by the many levels of training the entire system has endured. It may ask on the onset “What do I need to do now?” The system has been structured with a “breaker” lever which when pulled will discontinue any and all type of power to the system. The system also self-trains itself with operational data that does not involve any patients but just the overall operation of the system. The operation of the system utilizes checkpoint loops to continue set a data point in case the system needs to backtrack to a prior step. The checkpoint loops are also intermittently deployed to check for any errors. The system is built to self-correct and as more specific training is compiled for both operational procedures, steps, instructions and patient procedures, specific loop algorithms were written to compare successful results to unsuccessful results and note the differences in the operation of the system. This is how the system learns about its own operations. Computer software programs that are specific to some of the hardware components are labeled as third party software and are used in the MedsApp system. Some computer software programs as well as all types of algorithms (operational, step by step, learning, and forecasting) were also obtained from third parties. The algorithms and computer software programs can be downloaded from the internet for a fee. Some of the downloads come with instructions for computer programmers to manually revise some of the software code in the computer software programs as well as the algorithms. The system can also revise the computer software languages “computer software code” itself.

MedsApp data. The MedsApp data is in two separate forms, one for the operation of the system and the other for patient data both training and data for performing a physical autonomous medical procedure. This disclosure will concentrate on the patient data. In an aspect of this new disclosure, two different sets of data pools are utilized with MedsApp patient data, real time (in the now) and archived data accords in the past. The first data pool is the real time data of patients images, distances of body points to other different body points, and distances of patient body points to reference points on a MedsApp surgical operating table as well as data from UMMDA. This data is in the now called real time. The second data pool is the archived data of patients from all past medical physical procedures and its training. The archived prior patient body component images, archived prior distances of patients body parts to one another, and the distances of the images and the patients body parts are referenced to reference points on a MedsApp medical surgical table. The difference between the two data pools (first real time data pool to second archived data pool) is called the MedsApp Effect difference/Effect differential.

In an aspect of this disclosure, mathematical equations are converted into computer software programs and algorithms which fill in unknown data for training purposes.

In an embodiment of this new disclosure, mathematics such as algebra, sine, cosine and tangent (angles of triangles in the space of the surgical physical procedure/trigonometry), equations, ratios and proportions, interpolation, extrapolation, regression, averages and weighted averages, constraints and logic, and probabilities and statistics all work singularly and in combination with each other to determine and train data that needs to be gathered. In an example of this disclosure, integrals fill in areas of data not known. As an example of mathematical equations assisting with MedsApp data training, if a patients data is outside of all the data that has been trained for gallbladder removal, mathematical algorithms must be utilized to fill in gaps of unknown data. If the archived patient data base of gall bladder removal of the 400,000 patients has body weights of between 98 pounds and 227 pounds and the heights are between 5 foot 3 inches and six foot five inches. This is the gallbladder archived data-pool for MedsApp. What if the patient named Sammy is 360 pounds at 6 foot seven inches? Sammy falls outside the gallbladder data pool. In an example of how math is a tool in autonomous physical medical procedures, simple measurements are taken of Sammy's gall bladder with corresponding imaging and video, of other body components (bile duct) and that data is extrapolated, and compared in averages to fit into the gallbladder archived data pool. Mathematics must be used to perform the physical medical procedures by extrapolating data from data pools to perform self-directed physical medical procedures successfully.

An example of the training of algorithms in an aspect of the MedsApp system. The following is a basic example of a training algorithm performing step by step procedures. The MedsApp maintains a database of many different sub types of data with four main data types: (1) images and video that are normal as opposed to abnormal, (2) distances of reference points on patients, tables, robotic arms (with and without tools and devices) and walls that are normal as opposed to abnormal, (3) movements that through thousands of past archived successful procedures on subjects are normal (within the standard deviation), and (4) end results of what the end result should look like in all four prior forms-images, distances, movements, and end results. Some data points must have at a minimum of 40,000 data points to compare the new data (actual real time procedure and or application) to the old data (former data obtained, learned and archived by the system).

The MedsApp system is programmed to tell itself if the appropriate level of training and testing has been achieved for autonomous self-directed physical medical procedures and applications without human operator intervention. In an aspect of this disclosure, if the appropriate levels of training have been reached, if requested, the MedsApp system alerts the user (hospital, doctor, management of a healthcare facility via lights, verbal or in electronic correspondence) it is ready to go on its own. Milestones for training and testing may be the amount of successful procedures and tasks have been completed which differ in various physical medical procedures and noninvasive applications. The final step initially, in summary, is to train the autonomous MedsApp system to operate at a later date without any human intervention. The final goal was to implement the data obtained through training and testing to automate those trained and tested applications and procedures without any monitoring, guidance and or revisions by any human beings. As more training data is completed, tested for accuracy and archived, algorithms and machines will perform more self-directed physical medical procedures and applications autonomously. The first noninvasive application that were performed using algorithms and machines are physically performing tasks of testing oxygen levels of patients whereby other more sophisticated training will be required such as tracking and learning of heart surgery training for heart surgeries. The training and learning processes utilize many different types of applications, machines, devices and components. The algorithmic training, learning algorithms, forecasting algorithms and their associated computer software programs utilize many different components. The embodiment of this new disclosure is the training to enable algorithms and their associated machines to perform all physical medical procedures being autonomously and self-directed without any intervention or monitoring from medical professionals or human beings.

Self trained through hybrid algorithms. Algorithmic self-training through robotic assisted procedures whereby the software of the robotics trains the algorithms called reverse algorithmic training.

Description of MedsApp Software. Before any medical procedure or medical application can take place, there needs to be a patient (human or animal) but more importantly, there needs to be data associated with that particular patient allowing the MedsApp to make some initial decisions. This is where the new technology showcases its autonomous features.

The system starting points. The intake of a patient and the data gathered through all the patient intake applications and some intake invasive procedures such as oxygen level testing and bodily fluids (blood testing that requires a pin prick) through the UMMDA will be evaluated and MedsApp will determine through all the MedsApp patient intake applications what are the next steps. The UMMDA is a pending USPTO and PCT application from 2022.

In an aspect of this new disclosure, the MedsApp trains the system to evaluate and learn about patient data and then after evaluation inform a patient that a physical, a yearly checkup, a procedure should or must be performed immediately and to list in a text (or printed for the patient) and or to verbally speak the reasons for its decision during an interaction between the patient and the MedsApp via a chat bot.

Autonomous Patient Intake System. Before an autonomous successful physical medical procedure can take place, educated decisions must be made by the MedsApp. The only way in which the decisions can transpire is with the compiling of many types of data regarding the patient. This step must take place first and the data be compiled for the MedsApp to make the best decisions regarding the patient. The manner in which MedsApp intakes a patient is by an autonomous system. The following sections outline how an autonomous patient enters the MedsApp system “Intake”. For purposes of this disclosure, intake of a patient is defined as a new patient (person, animal or plant) that is entered into the MedsApp system whereby electronic data folders are created and shared throughout the system for machine learning purposes. The folders or “patient folders” contain many sub files of soft and hard data. Hard data of a patient are bodily fluids, cells and solid matter from a patient such as stool samples and skin for biopsies. Soft data of a patient is any type of electronic patient data that is stored in a computer storage device such as external images and verbal conversations.

MedsApp Patient Intake. In an embodiment of this disclosure, the MedsApp is a complete autonomous self-directed application for the intake of a medical patient. This is the point where the MedsApp becomes the actual doctor of the patient. All patient data also becomes data for training purposes. For purposes of this new disclosure, “MedsApp intake of a patient “isn't a treatment or procedure itself, it's the chatbot and ambient listening “MedsApp patient correspondence” and initial testing of patients vital signs. It's a non-invasive MedsApp medical application. The patient is in the care of the MedsApp as opposed to a doctor. The MedsApp patient intake process includes but is not limited to basic patient information such as birth date, contact info, medical history, reason for visit (symptoms, concerns, yearly patient checkup), vital signs which may include obtaining body temperature of patients, testing of patients heart rate, patients oxygen level testing, and patients weight.

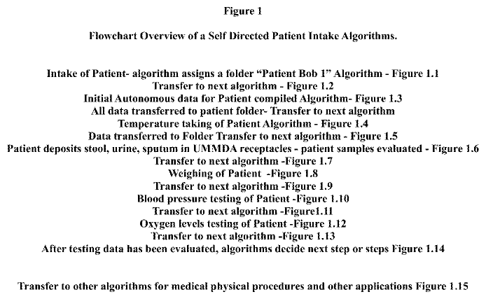

In an embodiment of this disclosure, intake patient data such as blood samples, urine samples, stool samples, and sputum samples are tested by UMMDA whereby the patient can deposit such bodily fluids into UMMDA via patient receptacle manually (see FIG. 1.6). The testing is autonomous in real time. Or, a robotic arm can gather bodily fluid sample and deposit the sample into the UMMDA receptacle. The robotic arm with a receptacle is placed under a patient to catch excrement and or urine. Missing the receptacle will cause the system to state in a forceful tone to re aim and then wash up thoroughly.

After the initial patient information and vital sign testing, the MedsApp has some information to start the patient health evaluation. This starts the next phase of patient testing. The next step is to test the patients bodily fluids utilizing the Universal Multi-Purpose Matter Detection Application “UMMDA”. The UMMDA is also an autonomous self-directed apparatus that detects a patients bodily fluids such as sputum, blood, urine, stool, and some tissue also known as patient sample data. MedsApp drones and robots are available to gather the samples if the patient is a paraplegic or is unable to place their finger in the UMMDA receptacle and assist with a pin prick for blood testing, spit into the UMMDA for sputum testing or unable to urinate into a UMMDA receptacle for urine testing. FIG. 1 is an algorithm of the intake of patient by the autonomous MedsApp system.

MedsApp Patient Intake Data History. The first part of most medical applications is the intake of the patient whether it be a person or an animal. Before any MedsApp new training data can be compiled on behalf of the patient and utilized by any MedsApp system, a new patient folder located on a MedsApp storage device must be formed and then populated with a full history of the patient. Patient data and history for purposes of this disclosure includes all geographic areas the patient lived before and during the patient intake procedure as well as medical data from themselves as well as all family members. Medical patient (medical patient or patient is defined in definitions) data for purposes of this new application includes but is not limited to any and all data that can be archived electronically, written, verbal, coded in computer software language (including qubits), forecasted data and mathematically obtained. Mathematically will mean any data that can be gleaned by mathematical equation, statistically, or by using probabilities and derivatives. Numerical analysis can also be used.

Patient Data explanation. Data prior to patients birth will mean any data that can be complied before the patient was born. Such examples would be parents health, grandparents health, and geographical area before the patient was born. An example would be if the patient was born in 2020 and there was a health alert such as the Flint, Michigan water issue from 2014 to 2019. This type of data will be referred to as environmental/geographical data. Another example would the plumes emitted from the east Palestine train derailment in Ohio. Data after patients birth will mean any data that can be compiled the day of and after the patients birth. Such examples would be surgeries, vaccinations, sicknesses, lifestyle data (smoking exercise, alcohol intake), or data such as driving an older car with fumes (1960s) or had exposure to the 911 disaster in NYC “911 dust”. Data affiliated with patient will mean all data that can be compiled. This data includes but is not limited to information and data from patients family, neighbors, friends, all medical data, or short term personal relationship affiliations. Such examples would be data of peoples health in all patients living areas (more than one college roommate became unhealthy for a period of time). All patient data will be archived in MedsApp computer databases which for purposes of this disclosure will also be defined as any type of data stored on quantum computers, quantum computing devices, computer processing devices, computer processor store devices, all future technologies regarding computers such new types of computer processors, new types of data (in space outside of earth's atmosphere) learned by machine learning algorithms in the new disclosure, and forecast events using all data (patient, learning and forecast). For training purposes, when a patient has had a meeting with a MedsApp chatbot professional (in this application, the medical chatbot professional can include computer software devices that performs, runs on and executes algorithms such as the MedsApp intake application), there are many types of data that will be used in this new application. The data can begin before the patient is born such as data from both biological parents as well as non-biological parents, grandparents, adoptive parents as well prior birth data. Most likely but not at all times this patient data must be manually entered into the MedsApp databases whereby the data may be verbal or in hand written on notes before computers were widely used.

Post-physical medical procedures data gathering from patient and all other data. The data obtained will be data from before the physical medical procedure, during the physical medical procedure and after the physical medical procedure of which may be cells from the patient and well as tissue and bodily fluids which can be stored in a patient intake hard data “matter” bank. There are four types of patient data for purposes of this new disclosure. Data prior to patients birth, data after patients birth, and data affiliated with patient and data learned from the physical medical procedure application. As more training data is completed, tested for accuracy and archived, algorithms and machines will perform more self-directed medical surgical procedures autonomously.

Public Domain Medical Data and unaffiliated data. The MedsApp will also gather all publicly disclosed research and third party publications to add to the MedsApp database and subsequent MedsApp patient data. This data could be from but are not limited to the internet, trade publications, the media, interviews on radio, the television, a computer streaming news, data from generative ai (tripled checked for being factual to other sources before using), podcasts, or obtained from private databases for a cost and an agreement with whom owns the private data. Ancillary Patient identification data are also used in MedsApp. DNA, bodily fluids, Iris, finger print, toe print, and facial identification as well as have a unique pattern and structure when used all together or used separately will recognize patients for identification and past medical records. This type of information will be used before, during and after any procedure or application has been performed by the MedsApp. The patients voice will also be used for data gathering. Some patients after surgery can elect to have a chip inserted in their bodies for identification purposes where some medical and identification data from the MedsApp data for them personally is stored. This can be used on the battlefields for identification of enemies as well as soldiers.

The Autonomous Self Directed MedsApp patient intake. The intake of a patient named Bob is an example of a first step where MedsApp gathers information about Bob before any treatment allowing the system to safely and correctly treat Bob if needed. Regardless of whether Bob is a new or returning patient, the system will gather data available through public means, data folders the system has accumulated from other subjects similar to Bob and compiled specifically regarding Bob, and after obtaining more information about Bob, the system will form a treatment procedure if needed and next steps for Bob. The system will gather all information in the MedsApp databases regarding the following: geographic area Bob lives in, lived in and what Bob was exposed to in the past and if any information stands out such as statistically more people develop throat cancer in that town where Bob lives. The system will gather as much information from Bob through the use of ambient listening technology and chatbots. The information requests of Bob by the system, are any health records that can be scanned, family health data, pre-birth, and post birth data Bob can provide the system. And finally, the autonomous self-directed system will start the process of intake patient applications which includes but are not limited to: Bobs blood pressure, body temperature, sputum testing, urine testing, blood testing (all three from UMMDA testing apparatus), body temperature, an entire scan of his skin, and oxygen levels.

After all Bobs data has been autonomously entered into his data folder (MedsApp can also scan his physical paperwork into his folder and read and organize that data), other patient data similar to Bob is then evaluated and compared to Bobs. The system will now search its own databases, the internet, private healthcare databases it has access too, for other patients similar to Bob. The data searched for other patients includes but is not limited to: age, race, gender, weight, height, similar health data and that of all other associated patient data similar to that of Bob.

If the system determines that Bob is healthy through data evaluation, the system informs Bob to come back next year for a checkup or make an appointment for a MedsApp.ai bus to travel to bobs residence for an autonomous medical robotic checkup. If through data evaluation, something is abnormal, the system informs Bob of a health issue and evaluates the next steps.

After intake of a patient, the MedsApp system acts as a doctor, doctors support staff, and clinicians through robotics, drones, algorithms and chatbots.

MedsApp is autonomous and has many forms of data training, evaluation, and step by step instructions. The system utilizes algorithms for every facet of intake of a patient, any procedure upon a patient, or all-encompassing of data gathering for a patient whereby all facets are autonomous directed by algorithms. For every movement, procedure, and decision, during the patient intake, the MedsApp learns through algorithms. The MedsApp learning at the beginning of the training of the algorithms was by a combination of the human hands, creating new algorithms and recalibrating old algorithms. The new algorithms are able to self-train data instead of relying on human data entry professionals to enter the data into the computer storage devices by hand.

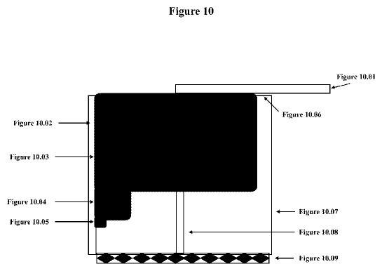

In an aspect of this new disclosure, the system is able to process normal and abnormal cell data evaluation in real time utilizing UMMDA or other MedsApp detection devices. UMMDA is also used in medical facilities to detect pathogens and harmful microbes before, during and after any interaction with a patient through MedsApp. In an example of MedsApp data evaluation, MedsApp will obtain the following data, and the score rating will be one out of 100 for gallbladder removal. The first test 1—Blood test (UMMDA) and stool test found gallstone matter. 2—MedsApp cameras detect bloating and distention 3—MedsApp detects jaundice. 4—Patient complains of abdominal pain, nausea, digestive issues, fever, whereby a robotic ultrasound was directed by the MedsApp and was found to show inflammation. At this point, MedsApp will perform a self-directed laparoscopic procedure to remove the gallbladder which will utilize, past patient gallbladder images (could be over 10 billion) to compare to every 1/10 of second of movement, measurements from markers (could be billions), while during the procedure, a minimum of 7 cameras are used, a surgical cutting tool to make an incision in the patients abdomen/pelvis, a color test to determine if the gallbladder does not look like others in the database (may show some disintegration). In an embodiment of this new disclosure, the MedsApp requests or demands a gallbladder procedure be completed based on a MedsApp patient procedure score. The MedsApp procedure demand score ranges from 1(MedsApp will monitor the patient) to 100 (MedsApp demands it must be done). A patient procedure score is based on the following scale: A score of 100 being that the procedure is needed, while a score of 1 is the MedsApp will monitor the patient. Any and all patient data compiled from the MedsApp adds to the score. The MedsApp may request an autonomous visit for a checkup by an autonomous MedsApp bus. Depending on the type of procedure and a score above 50.1, more testing may be required. The MedsApp assigns a “procedure demand score” from 1 to 100 which determines if a procedure should be performed. In this particular example, a score of 50.1 requests more testing for the removal of a patient's gallbladder. The following “procedure score data” was compiled from a patient utilizing MedsApp. From ambient listening MedsApp device, stomach pain was compiled as a positive score of 7.2 (seven point 2). MedsApp images compared the jaundice of the patient to 400,000 other patients in the database and scored an 8.7 (eight point seven) for having more than normal jaundice. The total score thus far is 7.2 plus 8.7 equals 15.7 (fifteen point seven). This score does not reach the threshold of performing a gallbladder removal. The MedsApp requested a UMMDA stool test which the score was 37.9 showing gallstone matter. 7.2 plus 8.7 plus 15.7 equals 31.6 (thirty-one point six). This does not meet the threshold of performing a gallbladder removal. MedsApp requested a UMMDA blood test whereby results of an infection was detected and given a 61(sixty one). 7.2 plus 8.7 plus 15.7 plus 61 equals a 92.6 procedure score which reaches the threshold of demanding the gallbladder be removed. In an embodiment of this disclosure, MedsApp system self directs algorithms to perform a medical procedure without human operator physical assistance. In another example of an autonomous MedsApp physical medical procedure, if the gallbladder was determined by MedsApp to be removed by a directive from the MedsApp one of the next steps is to access the gallbladder procedural data. A further step may be the searching of gallbladder data in archived folders and ancillary data that is similar to patient such as weight, age, procedure score, what is their lifestyle which the MedsApp listening technology will evaluate and give a lifestyle score with questions such as use of marijuana, cannabis cigarettes, chews and or smokes tobacco, an alcohol drinker, level of exercise, level of stress from family, job and financial stress to list a few of many MedsApp questions. If the UMMDA scans the rooms for microbes and has a positive hit, the MedsApp may require a misting of biosurfactants before the MedsApp medical procedure is started (see FIG. 10 of a biosurfactant tank). The next step could also be searching other databases outside the MedsApp archived data pools as well as the internet if a datapoint is outside of the norm such as the weight of the patient.

In an aspect of this disclosure, after all intake procedures and applications have been completed, one of two things may transpire. Patient is healthy and the MedsApp will see them in a year or the next scheduled visit, or MedsApp has found something abnormal or irregular. Depending on the type of irregular finding, it could be determined through the intake testing that MedsApp needs to complete more testing procedures or put patient under watch weekly. One aspect is to have the MedsApp take a look internally of a patient by a MedsApp small roaming self-propelled vehicle, or the MedsApp will open up (make an incision) on a patient for evaluation and prepare for the removal of an organ such as a gallbladder. The two choices will be discussed later, whereas the MedsApp software will be discussed next. The third choice is to put patient under a high level watch or search databases for other options.

In an aspect of his disclosure, simulation software is utilized in the training of the MedsApp system. Simulation can be used as a component to training the MedsApp. The simulation software mimics tracking movements of both machines and human operators for training purposes. The MedsApp tracking technology is where the system tracks through various sensors the movements of all movements during a physical medical procedure. The tracking software is used to enable the machines to mimic the movements by both human operators and the mechanical arms to simulate the movements and further transfer the simulated “mimicked movements” into training data. MedsApp simulation software utilizes quantum technologies as well as quantum computing.

MedsApp checkpoint loop(s) is a system software application where the state of a step is periodically saved, allowing it to resume from the last saved state in case of an interruption or failure. This is particularly used in long-running applications to prevent loss of progress and improve fault tolerance especially in physical medical procedures and intake of a patient through ambient listing technology. The MedsApp has many checkpoint loops for each algorithm called a MedsApp checkpoint loop correction or when a next step has been made but in real time new data was learned that needs to change the direction of the procedure.

MedsApp.ai Hardware Components. The methods of physical medical procedures will be described starting in section 130, at this juncture MedsApp hardware will be described. The components of the MedsApp system are sensors, tools, devices, image enlargement devices, instruments, mechanical arms, robots, drones and adjustable operating tables all being self-directed by algorithms to achieve a medical goal without human operator intervention. Hardware also consists of new technology in processing chips which is described in this specification. The entire system, its components, its methods, its algorithms, its hardware components, its computer software components, modules, control units (both software and hardware), biosurfactant applications, stationary and mobile mechanical arms, mobile vehicles, tools, devices, instruments, 3D printers, UMMDA, (replaceable cartridges for UMMDA), quantum computers, training programs and procedures are named MedsApp.ai or simply “MedsApp”. MedsApp is a system that operates without any human operator assistance, intervention, or interaction. At times MedsApp may also be referred to the “system”. The final goal of the MedsApp is to be trained to allow the system to perform physical medical procedures and physical medical applications on its own without any human operator help, human operator intervention or human operator monitoring. MedsApp modules (the decision engines included in the systems suite of algorithms) automate robots and drones with medical devices (and tools) attached to mechanical arms with types of MedsApp sensors (e.g., optical, LiDAR, pressure, thermal), imaging sources (CBCT, MRI, ultrasound. magnetic resonating machines) or be refitted by MedsApp helper drones (tethered and untethered) and robots (tethered and untethered) to assist in the MedsApp self-directed medical procedures. The MedsApp safety mechanisms are automated emergency stop control devices and mechanisms by the algorithms and set by the algorithms, for confidence thresholds, real time monitoring of algorithms, and patient non-voluntary and voluntary responses. The algorithms and software programs associated with the sensors, tools and devices that operate the sensors also learn through machine learning.

In an embodiment of this disclosure, processing chips compute and calculate the computations in the MedsApp systems. They include but are limited to: CPU (Central Processing Unit/Units) that are for general-purpose “brain” of the computer software devices. GPU (Graphics Processing Unit/Units) are large parallel processors that provide graphics for the MedsApp systems. TPU (tensor processing units) NPU (neural processing unit chips), and machine learning accelerators are chips specialized for neural-network and machine learning workloads. DSP (Digital Signal Processor/Processors) are optimized for real-time MedsApp applications such as cameras, audio, video, and sensors. FPGA (Field-Programmable Gate Array/Arrays) are reconfigurable MedsApp hardware where specific MedsApp applications and logic can be reprogrammed after manufacturing of such. ASIC (Application-Specific Integrated Circuit/Circuits) are custom chips built for one main MedsApp task such as video codec, crypto engine, or skin scans. MCU (Microcontroller Unit/Units) are small chips with CPU plus memory plus peripherals for embedded control of robots, sensors, and UMMDA. SoC (System-on-Chip/Chips) combines CPU, GPU, memory controllers, radios, etc., on a single chip such as MedsApp screens, touch screens, phones, tablets, and IoTs. Storage and memory chips hold and store the data for MedsApp. SRAM (Static RAM) is used for CPU caches in MedsApp. DRAM (Dynamic RAM) is a system main system memory device such as DDR4/DDR5 modules utilized by MedsApp. Flash storage or NAND Flash storage is a non-volatile storage used in SSDs, USB drives, eMMC, and UFS in the MedsApp system. EEPROM are Electrically erasable ROM which stores small amounts of configuration data. ROM, PROM, and EPROM are read-only or one-time/limited-time programmable memory utilized by MedsApp systems. NVRAM, MRAM, ReRAM, combine speed of RAM with non-volatility for the MedsApp systems. MedsApp system storage controller chips which are controllers inside SSDs and disk drives that manage how data is stored and retrieved.

This new application utilizes new computer processing chips. The new computer processing chips are defined as new CPUs, GPUs, AI Chips, DSPs (Digital Signal Processor), Application-Specific Integrated Circuits (ASIC) Enhanced Capability Chips, Specialized AI (AI being Artificial Intelligence) processing chips, microchips, integrated Circuits (ICs) and quantum computing processing (processor) chips. These new chips as opposed to the older chips handle increased performance while reducing energy consumptions. Advancements in computer processing chips (GPUs and CPUs) scale up performance. Some of the processing chips utilize cooling systems to reduce heat. Some of the new chips will handle quantum computing applications. Chips can be, digital, analog, quantum computing chips, or combinations thereof. Some chips are specifically made for quantum computers and operate only with qubits.

In an embodiment of this disclosure, one or more integrated circuits selected from: general-purpose processors, graphics processors, digital signal processors, microcontrollers, application-specific integrated circuits, circuit boards, field-programmable gate arrays, and memory devices including volatile and non-volatile semiconductor storage are utilized by MedsApp.

In an embodiment of this disclosure, memory and storage chips and devices are utilized by MedsApp. They include but are not limited to: Relational (SQL), MySQL, PostgreSQL, and Oracle. NoSQL, MongoDB, key-value stores, Redis, wide-column (Cassandra), graph DBs and Neo4j, NTFS, ext4, and APFS. Network file systems such as NFS, and SMBs. Cloud and object storage such as Amazon S3, Google Cloud Storage, and Azure Blob. Data warehouses and data lakes such as Snowflake, BigQuery, and Redshift are used. Data lakes such as large, raw data repositories are also used. In-memory storage and caches are used. RAM-based storage such as Redis and Memcached are used for fast access. Distributed logs and queueslare used. Apache Kafka and Pulsar append-only logs as well as event streams are used with MedsApp. Version control systems such as Git and Subversion which are specialized storage for source code and revisions are used by MedsApp. MedsApp Physical archival storage such as Hard drives (HDD), solid-state drives (SSD) are used for long-term data storage and archives.

In an embodiment of this disclosure, MedsApp storage can utilize every and all storage technologies which include blockchain.

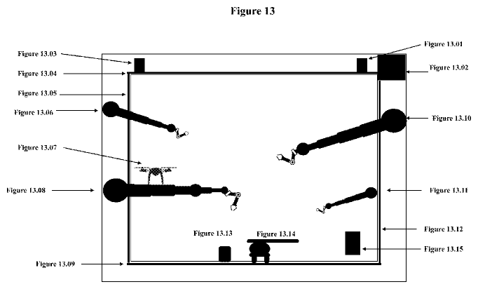

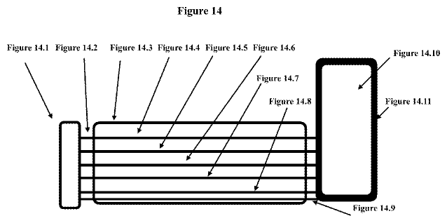

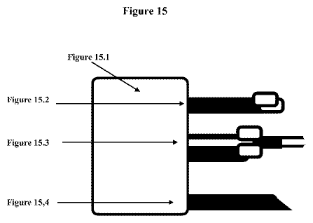

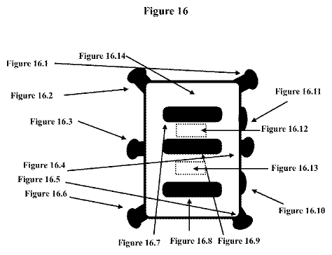

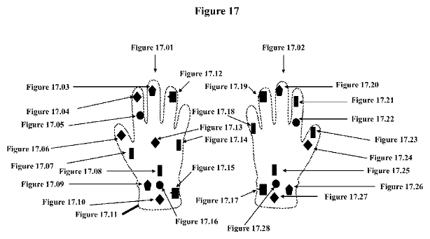

MedsApp system deploys robotics in the MedsApp operating room to perform both training procedures and training applications as well as performing physical medical procedures and medical applications without intervention from human operators or clinician operators. The MedsApp robots operate in many different capacities in self-directed procedural operations. The MedsApp robots operate with and without mechanical arms that maintain several different types of tools and devices some of which are listed in this disclosure. FIG. 14 and FIG. 13.08 is a mechanical arm used by the MedsApp system. FIGS. 14, 15, 16, and 17 and FIG. 13.11 shows mechanical arms with platforms where attachments can be attached such as tracking devices, motion devices, listening devices, measuring devices (with reference points on patients and or surrounding room), and lighting devices “instruments” are all interchangeable. The instruments work singularity, in combination with one or more, or all together to record everything going on during a training procedure or a surgical procedure on a subject.

Tracking of medical instruments, medical devices, measurement devices, and positioning devices can be embedded with other different types of devices whereby tracking devices also have secondary tracking devices.

MedsApp tracking signals utilizes wired tracking MedsApp devices whereby the data from any tracking device in the MedsApp procedure room can be connected by wire (cat 5 wire) directly connected into a computer software device. Signals and data transfer can also be transmitted wirelessly whereby a computer software device with computer software programs transfer that wireless data into data. The data then can be transformed into visual, verbal, 3 dimensional (x, z and z axis) or computer software languages. Algorithms processes all types of data which includes but is not limited to: physical movements in 3-dimensional, verbal, text in all languages, another computer software language, or a combination of them. Computer software devices are laptop computers, cellular phones, tablets, servers or any computer software device that can store data. This includes quantum computing applications and quantum computing devices. Cords attached to a clothing worn by a medical staff professional for training can be attached to an electrical outlet to provide power to tracking device gloves. This application could also be battery operated with the cord being attached to a battery in the clothing worn by a medical staff professional for training.

MedsApp Robotics are defined as any and all actions and steps a mobile or stationary robot may perform. Vacuums are used with MedsApp to attach to body parts and move those body parts in place. The releasable suction cups are vacuum operated. Magnets can also be used in certain MedsApp applications where magnets can be placed in the body cavity of the patient during a surgical procedure. This helps pull the magnetic tools towards its destination. In an embodiment of this disclosure, MedsApp Mechanical arms mimic movements of human arms, hands and fingers for both training purposes and autonomous physical medical procedures. They can operate by beams, hinges, hydraulics, mechanical devices, actuators that self-direct the arms, hands, and fingers by algorithms and computer software programs. The beams can have power wires within them. The fingers can be equipped with pressure detection, texture detection, color detection or other type of detection devices and or tracking devices needed in training and medical procedures. Fingers, hand, and wrist press on tracking devices (with glue and or adhesive) for physical medical procedures that can be tracked by wireless data transfer with the tracking devices being applied directly to the hands and fingers of human operators.

Scanning of images are also used by MedsApp. In an embodiment of this new disclosure, the action of scanning of images before, during and after procedures is used. An example would be the images compared to past images that were part of a successful procedure. A further example would be if a replacement of a kidney procedure would compare scans of past images and or recordings of successful kidney replacement procedures. In an embodiment of this new disclosure of this new disclosure, scanning by lasers would provide imaging of present medical procedure images from comparison of images from past procedures. The images would be compared by algorithms that have access to databases of patient data.

In an embodiment of this disclosure, MedsApp utilizes real time diagnosis of tissue, muscles, tendons, organs, or any type of matter from a subject. The following matter detection devices used are ultrasound devices, electron microscope devices, image enlargement devices and UMMDA all self-directed by the system. The matter from the subject is obtained by machines that are self-directed by algorithms. For training purposes, a manual method of obtaining a sample by hand can also be used as opposed to obtaining a sample by self-direction. For physical autonomous medical procedures, the 3 detection apparatuses can be utilized in single form or in combination with each other and can detect abnormal cells and abnormal tissues in real time by also utilizing quantum technologies. The system through its machines and algorithms make real time decisions on subjects such as detection of an abnormal looking organ. All three detection apparatuses may work as a single unit, separate or in combination with each other to verify the accuracy of matter detection results.

The procedure of mimicking movements for training purposes in this new disclosure consists of both software and hardware components. It utilizes lasers, lights, tracking devices, cameras, simulation software, dyes, black lights, and mirrors that track all movements of both medical professionals and mechanical arms that perform medical surgical procedures.

In an embodiment of this new disclosure of this new disclosure, mimicking for training a procedure works in many different ways. Whether the physical medical procedure is using a mechanical arm or mechanical arms, a single medical professional, a group of medical professionals, or a single or group of medical professionals utilizing mechanical arms, robots and drones, the procedure mimicking movement application can be used. The autonomous physical medical procedure operation requires room for cameras, image enlargement devices, medical tools and device as well as mechanical arms whereby the procedure must be recorded in 3 different manners 1—tracking devices that are attached to many areas on the hands and fingers of the medical professional staff, areas on the medical tools, areas on the mechanical arms as well as the subjects body internally and on its skin. Headset googles track the movements, emitting digital, radio and light waves from the tracking devices are recorded via light waves, lasers, and x y and z tracking software.

MedsApp also utilizes an electron microscope system in the MedsApp. This is a secondary detection device to the UMMDA. Another way in which matter samples from subjects can be tested by not using the UMMDA is the placement of matter from the patient by way of robot and or drone on a stage with an electron microscope. The application works by either robots and or drones that maintain mechanical arms with medical devices that obtain matter samples from patients that are directed by algorithms. The samples are placed on the stage of an electron microscope through an open and closed door that uses high beams of electrons instead of visible light to illuminate the matter sample whereby electromagnetic lenses are used. The application replaces image enlargement devices for beams of electrons on sequestered matter testing application on a stage. MedsApp image magnification devices can be any type of enlargement devices such as lenses, light microscopes, folded mirror lenses or any electronic or mechanical device that can enlarge the image of matter. Quantum computing technologies are also used with the MedsApp electron microscope. The entire process would be autonomous and managed by algorithms directing the electron microscope to have numerous superpositions evaluated at one time.

In an embodiment of this new disclosure, image enlargement devices can magnify images up to 1,000,000 times using quantum computing combined with image enlargement devices.

In this new disclosure, 3D printers are utilized with the MedsApp which are positioned inside the MedsApp operating room and are on standby. The system in real time determines if a device or tool part, screw, bolt, body component such as a heart value, knee or hip replacement is needed and can be made in real time inside the MedsApp operating room next to patient with exact measurements taken by robots from the patient for the construction thereof. The MedsApp artificial body parts and devices can be made semi inside the body using a miniature 3D printer after technology advances. In an aspect of this disclosure, quantum computing technologies and qubits are utilized with the MedsApp 3 D printer. The MedsApp AI construction of any type object of MedsApp AI utilizes quantum computing.

MedsApp electromagnetic tracking utilizes electromagnetic micro sensor which is affixed to the fingers (and palm) of a medical professional and or affixed to the mechanical fingers on a mechanical hand attached to a mechanical arm during medical surgical procedure that is tracked by algorithms. In an embodiment of this new disclosure of this new disclosure, MedsApp electromagnetic tracking utilization utilizes quantum technologies as well as quantum computing. This produces 3 dimensional images of a patient, over 4 with quantum technologies.

MedsApp produces 3 dimensional images of a patient utilizing many electromagnetic sensors, whereby this new application can produce 3 dimensional images (also using simulation software) of a patient, a patients body, a section of a patients body, or a specific organ of a patient. These images are tracked and archived by algorithms.

Powering the MedsApp System. This new disclosure can operate by any power source which includes but is not limited to electric, battery, hydrogen, natural gas, nuclear energy (fission/fusion), water power, solar power, thermal power, combustion, and gravity. Some of the other power sources also includes pneumatics, hydraulic systems, hydrogen, springs, wind power, magnetics, chemical, pressured air or gases, and fluid technology.

Gravity may assist a MedsApp medical procedure. Other than the physical effect of gravity on hardware components of MedsApp, the system can make use of or be influenced by outside forces (not human) by heat, cold, temperature changes, currents such as wind, fluid or electrical, heat transfer, electromagnetic. Physical applications refer to the practical uses of principles and theories from physics in real-world scenarios. Gravity may be required for a specific physical medical procedure on a patient strapped to a MedsApp operating table. Utilizing a rotating table, gravity is used to have organs become free from body cavities by the weight of the organ due to gravity. The space made by gravitational pull is enough for the MedsApp robotic arms to perform physical medical procedure by having more room between the organ and the body cavity. Human medical staff and doctors find the rotated body of the patient difficult to work on while the autonomous robots don't mind at all. The amount of space required for the autonomous robots to perform a physical surgical procedure is very small while human doctors and their medical staff require more than 30 times the space. In an aspect of this disclosure, MedsApp operating tables can be turned upside down with a patient strapped to the table for ease of autonomous physical medical procedures.

In an aspect of this disclosure, MedsApp self directs miniature robot in cadavers for training purposes. MedsApp has devised algorithms for a robot to roam internally in a cadaver for purposes of being able to cause minimal internal damage in the animal or human body that won't affect the health of such. The miniature robot can be tethered that may cause damage to surrounding areas in a body cavity when used. When the MedsApp autonomous self-directed robot is released, placed, injected into the body cavity, data is tracked, marked, mimicked, measured and transmitted to MedsApp computer software devices and at times quantum computers. MedsApp robot can be injected though a small incision, or injected through the skin of the patient, or via a hypodermic needle or entered through the navel, anus, mouth, nose, ear, urethra, or anywhere else such as a cut already present on the patient. The miniature robot is called the MedsApp roaming robot. The MedsApp roaming robot can be tethered, untethered and powered by any power source listed in this disclosure. The MedsApp roaming robot can also be directed by magnetics with one force being outside the body and another being inside the body. The MedsApp also utilizes roaming autonomous nanotech (small) UMMDAs to self-propel internally throughout the body to gather data. The data obtained can be chemical, human body cells, images, living matter such as worms and parasites, gas, liquid, solids, matter size, color, or dead matter (was alive but is not at the time of detection). The robots may maintain miniature image enlargement devices as well as nanotech components to the UMMDA. The UMMDA gathers real time data with accuracy from matter on, inside or outside close to around the body such as matter attached to hair on the body. This robotic component system and operation can be programmed or can be self-directed to gather image comparison to other archived data. The robot can take pictures, video, and provide a lighting source for illumination. Training data is always obtained regardless of what, where or in what manner the data is obtained from any type of patient dead or alive.

In an aspect of this disclosure, an autonomous vehicle utilizes the MedsApp system. Many components of the MedsApp are installed in an autonomous vehicle. For purposes of this disclosure, an autonomous vehicle is a self-driving vehicle with no human operator input. The autonomous vehicle can be of any size but most of the MedsApp vehicles are larger than passenger cars. An autonomous vehicle which may be a converted bus, a converted tractor trailer or a large industrial vehicle such as a six or more axle tractor trailer trucks (semi-truck) with tandem trailer (a double tractor trailer truck). The MedsApp autonomous vehicle travels to different geographical areas whereby any person or animal can seek treatments from the MedsApp physical autonomous medical procedures by being placed by a Lyft installed on the MedsApp autonomous vehicle or the patient can enter the vehicle using their own power. The entire MedsApp autonomous vehicle (also known as the “AI Med Bus”) is not assisted, monitored, or guided by a human operator being whereby all internal procedures are also autonomous. Med Bus is equipped with wireless transmission devices to connect via cell phone tower or satellite to remote computer processing devices or quantum computers. The remote computer processing devices and quantum computer computing devices can be used with the AI Med Bus as well as the static AI MedsApp application. The computer software and processing devices and quantum computer computing devices can be combined to form a daisy chain or remain separated but operate together with the MedsApp as well as the AI Med Bus. The AI Med Bus can be powered by battery, rechargeable batteries, combustion engine, solar power, heat, gas (natural/butane/liquefied petroleum gas) thrust engine, wind, nuclear energy, or plugged into an electrical socket or a nuclear power plant. The AI Med Bus can also maintain a charging device with the charge being stored in power storage devices. The charge can be obtained by the motion (kinetic energy) of the AI Med Bus. If available, DWCS can also be used by the AI Med Bus. The AI Med Bus can also be powered by any other type of power source listed in this specification. The AI Med bus will also be equipped with a cryptocurrency machine to take payment for services performed on the AI Med Bus using cryptocurrency. The AI Med Bus will also dispense receipts for cryptocurrency payments, procedure printed receipts, emailed receipts or verbal receipts from the ambient voice to text/text to voice application or ambient listening technology.

This section explains quantum technologies and quantum processing methods utilized in MedsApp systems. In an embodiment of this disclosure, quantum computing technologies enable medical procedures to be more precise with faster real time data processing. MedsApp also enables qubit data to be stored in the cloud with easy access of the archived data. Quantum computing through MedsApp is also stored on blockchain. In another aspect of this disclosure, quantum computing is used for faster evaluation of MedsApp data, images, marker tracking, and MedsApp robotics to perform at a faster and more precise rate of action. Quantum technologies also supercharge predicting of future medical events in MedsApp systems. Forecasting and predicting events are built and designed on the same premise, what has happened with the data obtained from MedsApp, and what may happen. This is done from dot plotting events in a 3 dimensional space with mathematical derivatives written in computer software code to point out future possibilities. With quantum computing, the data can be evaluated in more than 5 spaces (superpositions) at one time. If the possibilities come true at a future time, that data is then trained and utilized by the derivatives to predict what else may happen in the future. Data from the UMMDA is entered into a separate derivative algorithm as well as a derivative algorithm from other aspects of the MedsApp as well as quantum computers.

In an embodiment of this disclosure, quantum computers are part of the MedsApp.ai system. MedsApp utilizes quantum computers to complete medical procedures and medical applications in a faster manner with more precision. A quantum computer is defined as a type of computer that uses the rules of quantum physics to process information for MedsApp. Instead of bits that are only 0 or 1 in classical computing, quantum computing uses qubits, which can be 0, 1, or both at the same time. This technology allows many possibilities in parallel and potentially solve certain problems much faster than regular conventional classical computers that utilize classical algorithms. Quantum computing utilizes superconducting qubit chips. Quantum algorithm processors, qubit processors, trapped ion systems, photonic quantum computing, neutral atom systems, qubits spun in semiconductors, qubit chip or trap photonic circuit, control stacks, transmon qubit technologies (up to 3 qubit transmon), Josephson junction circuits technologies are all incorporated in this new disclosure.

In an embodiment of this new disclosure, quantum computing performs self-directed procedures and applications in the medical, dental, dermatology, ophthalmology, cosmetic and veterinarian professions. The procedures and applications use the rules of physics (quantum mechanics) to test many possible options at once, which enables a MedsApp robot to train itself and learn best decisions faster and more efficiently for procedures and applications in the afore mentioned industries. In quantum mechanics, tiny particles (like atoms or electrons) can be shown in several states at the same time. In quantum computer calculations, states of quantum computing can reside in other states, or states within states. The substate could be in the 4th superposition or 5th superposition. Quantum computing is used in many MedsApp components including the algorithms, UMMDA, robotics, drones and 3d printing devices.

In an aspect of this new disclosure, the MedsApp system self-trains itself by self-directed machines, tools, devices and algorithms with some algorithms being quantum based.

In an aspect of this disclosure, MedsApp system utilizes quantum technologies, quantum computers, and qubits. Quantum computing is used for both training of data and performing a medical procedure and or medical application autonomously utilizing the trained data without requiring human operator/clinician intervention, guidance, or monitoring. Quantum computing is used to train the MedsApp system either singularly or in a superposition state.

MedsApp system utilizes computer software programs, classical algorithms, and quantum computing algorithms separately and in combination with each other for autonomous physical medical procedures. MedsApp algorithms use Tribrids which are combinations of all three computer software programs, classical algorithms, and quantum computing algorithms. Hybrid algorithms are defined as a combination of classical algorithms and quantum computing algorithms. MedsApp self-training implements both classical algorithms as well as quantum computing algorithms.

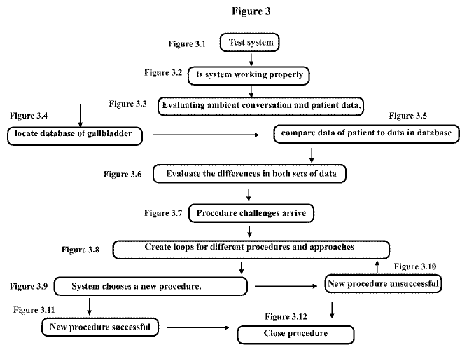

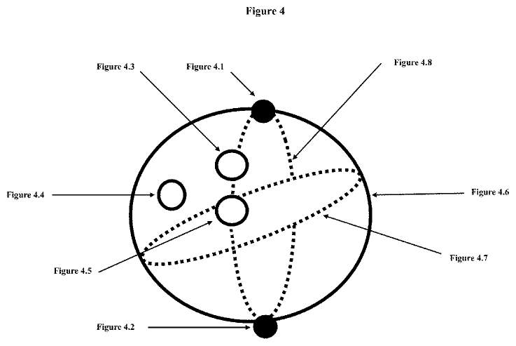

In an aspect of this new disclosure, present real time data of a subjects body component position is measured, referenced to other reference points and set on an x, y, and z axis forming a 3 dimensional space. When utilizing quantum computing, there can be more than a 3 dimensional space. The MedsApp has archived millions of images and their distances of one subjects body component to other different archived of several subjects body components. An example is the distance of 47,170 archived images of the bottom left third rib of those 47,170 different subjects to all areas of a gall bladder of those 47,170 subjects. The entire areas were mapped in 3 dimensional spacing and imaging. In an aspect of this disclosure, using quantum computing, 4 or more dimensional views can be created with exact measured spacing by utilizing the x, y, z, and k axes. In an example of this new disclosure, FIG. 4 is an overview of a quantum computing superposition. As opposed to classical algorithms which operate on a step by basis, quantum computing can operate on many different steps at one time. In yet another example of this disclosure, FIG. 5 is an example of the MedsApp system that is trained on billions of images that are from many different views with high definition images that show very fine images of human bodies and all their components. Quantum computing technologies enable the MedsApp system to perform with a higher degree of faster results with the evaluation of images by the billions in real time. Classical algorithms take longer to evaluate data. FIGS. 4 and 5 outlines how the MedsApp quantum technologies performs on many different levels at once as opposed to classical algorithms that perform calculations one by one. An example is if the MedsApp must complete a successful gall bladder removal, the MedsApp has been trained on correct images on every slight step and or movement that is made. FIG. 3 is an overview flowchart of a gallbladder removal. An example is pictures and exact areas where the gallbladders are located whereby trackers are imbedded in images of gallbladders for reference. The removal process mimics millions of videos that are archived in the Meds App databases from past gall bladder surgeries which run concurrently through quantum computing. During the procedure, every slight movement of a tool or device is mirrored by concurrently running videos and comparison images to correct slight procedures missteps or mishaps. Each procedure may produce more than 10 billion images and 10 billion videos that are compared to the actual gall bladder procedure which also produce 10 billion images and 10 billion videos in real time through quantum computing. Quantum computing allows the MedsApp to switch back and forth “bounce” between past successful procedures on many subjects to a present surgical procedure on one subject with a prodigious amount of evaluations at one time while calculating different subset levels of evaluations as opposed to classical algorithms.

UMMDA has been upgraded from the original USPTO filing to include quantum technologies in its detection methods whereby it performs faster for detection of matter in operating room surroundings as well as abnormal cell detection utilizing quantum computing.

In an embodiment of this disclosure, MedsApp quantum technology, quantum computing, and robotics accelerates cognitive architectures in MedsApp robotic medical, dental, dermatology, cosmetic, ophthalmology, and veterinarian applications. The MedsApp system utilizes quantum computing algorithms. Quantum computing machines return higher-fidelity results than classical algorithms. MedsApp quantum computing optimization algorithms give better solutions or better scaling for many medical procedures. Classical MedsApp algorithms can propose hundreds of options or step by step procedures whereby quantum algorithms provide thousands and thousands of data points into a higher dimensional space for analysis. Quantum data kernels are utilized by MedsApp for faster and more precise data evaluation. MedsApp deep machine quantum computing learning has completed tasks with more permutations to arrive at a specific goal with more outcomes for the patient. MedsApp deep machine quantum computing learning understands morphogenesis patterns which in turn enables the system to become more exact. Quantum computing algorithms also known as quantum optimization algorithms are techniques that use quantum computing to solve problems faster, than other possible step by step options. Quantum approximate optimization algorithm accelerates combinatorial optimization problems for MedsApp.ai systems. Quantum annealing is also another MedsApp method which evaluates and finds optimal solutions for complex optimization problems by deploying quantum mechanics. Q-tables, or Q-value tables, are used in training of data that finds a certain action in a given state, helping the MedsApp make a faster better decision in real time. Quantum computing algorithms are specialized algorithms that utilize quantum computing to solve challenging problems, which involve finding the best solution from a set of possible options in a faster manner. The quantum algorithms offer significant speeds over classical algorithms, particularly for complex problems in the medical, dental, and veterinarian industries. Quantum annealing is a quantum computing method that searches for and finds the optimal solution to complex optimization medical problems by leveraging quantum mechanics, such as superposition and tunneling. It is particularly effective for questions like “hey MedsApp—what is the next best step for a specific medical procedure for a patient”, allowing it to explore solutions from trillions of other data points efficiently than classical methods which enhances real time decisions for the MedsApp. Quantum computing in the cloud is a foundation of remote access to quantum computing resources over the peer to peer processors (also the internet), allowing MedsApp to run quantum algorithms without needing specialized hardware. The MedsApp quantum cloud loop is a system that utilizes quantum processing technology for secure data transmission and communication, often involving fiber-optic networks. These systems leverage principles of quantum mechanics, such as entanglement, to enhance security and efficiency in data transfer. MedsApp quantum cryptography secures data transmission by using the principles of quantum mechanics, which is unhackable by bad actors. It primarily involves quantum key distribution, which provides two parties with a secret key.

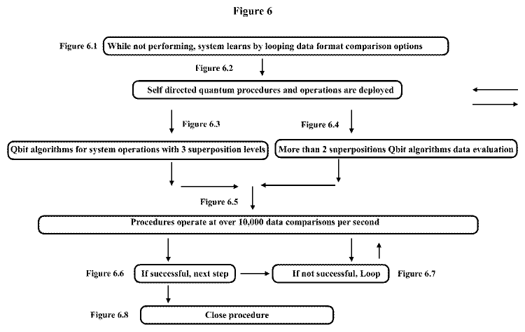

MedsApp quantum improved medical diagnostics refers to the use of quantum computer processing and quantum technologies to improve training, accuracy, speed of medical diagnoses, and procedures. See FIG. 6 for an overview of quantum superpositions. This approach leverages principles like superposition and entanglement to analyze complex data more efficiently than traditional methods, potentially leading to better patient outcomes. The MedsApp quantum training system is designed to simulate other quantum systems such as a human body simulating a touch of their toes utilizing quantum mechanics. This application provides more valuable data for the MedsApp training system. Image reconstruction and enhancement through MedsApp quantum computing provided precise data on a 3 dimensional level. In certain applications, a 4 dimensional space (and many more dimensions in other applications) is simulated (simulation software) by the MedsApp.

In an embodiment of this new disclosure, MedsApp quantum technology, quantum computing, and quantum robotics accelerates cognitive architectures in MedsApp robotic medical, dental, cosmetic dermatology, ophthalmology, and veterinarian applications. Quantum computing is integrated into the processing technology into the MedsApp mobile vehicles for physical applications. The addition of quantum technologies improves the performance of both the MedsApp hardware and software systems. The advantage of quantum computing is the reduction of computational time with more accurate data due to the operation in many superpositions at one moment in time. One such example would be the slightest motions of the MedsApp robots and their mechanical arm motions. With quantum technology, many robots are involved in a single physical medical task. MedsApp medical robotic swarming of intelligence where one robot assists others whereby, they make informed decisions about their tasks and goals as a whole. Deploying quantum computing applications allows the MedsApp robots to also create 3D maps of the patient which is required for tasks such as evaluation, inspection, and application of procedure at a faster pace. Quantum computing in MedsApp robotics provides faster and more efficient computation for real time guidance. In an embodiment of this disclosure, the usage of Grover's algorithm is utilized. The computational application was made public by Lov Grover in 1996. It is a quantum computing application algorithm that searches databases whereby “time to search” is compared to classical algorithms which proves that the application is much faster than the non-quantum conventional algorithms. When used in three dimensional spaces (three-dimensional Cartesian coordinates), the time to perform a task is decreased more than 100 times which in this case is the MedsApp cartesian coordinate robot. The algorithm utilizes each robot's location to the patient's body within a quantum state, where the location of each robot is utilized in a specific manner. By applying quantum superposition and entanglement, emerging quantum computing models can assess many problems at once with learned reasoning. In Quantum Reinforcement Learning (QRL) and Quantum Deep Deterministic Policy Gradient all these problems are learned from. An example is when the patient requires a biopsy, one robot evaluates and plans, one performs the biopsy, one monitors for problems and one provides sutures, and one provides the green light that no bleeding is current, and the final result is successful. This matters when a false loop is encountered, and unexpected obstacles demand quick re-planning and many robots with different quantum and conventional algorithms work together to solve the problem. The expedited capacity of machine learning and real time decision making is paramount in the medical industry for the benefit of the patient.

Quantum computing for training MedsApp robotics utilizes quantum processors and quantum computing as a powerful “MedsApp Q Training” and “MedsApp Q Systems” for its systems procedures. Classical algorithms self-directing a robot utilizes one by one steps whereas the MedsApp Q Training and Q Systems analyzes and searches through many combinations at once. In an embodiment of this disclosure, the quantum computing system makes it easier and faster for the robot and the entire system to learn things like safe motions, precise movements, and optimal strategies, especially in complex environments such as medical procedures and medical applications. This new technology and marker positioning enable more precise incisions in organs and body components.

In the MedsApp system, quantum computing and quantum processing acts as a smart optimizer for training procedure for robots and drones. The MedsApp robot has many more choices with the quantum computing technology to choose for possible motions and strategies. MedsApp utilizes the quantum algorithms to search those possibilities for efficiently, precision, with the patients health in mind highlighting “Best” decisions. The MedsApp robots train with the goal of safer, more precise, and more reliable medical procedures than the system could get with classical training alone.

In embodiment of this disclosure, quantum lasers are utilized to burn skin and tissue without surrounding damage outside of the area targeted such as other cells not targeted. Quantum medical imaging utilizes reflection devices, mirrors, and absorbing surfaces. The MedsApp quantum lasers are used by MedsApp robots to eliminate some cancers, moles, and skin imperfections among other invasive procedures as well as for measurements.

Quantum-enhanced magnetic induction tomography for spatial resolution imaging and quantum-enhanced (quantum technology imaging, quantum computer imaging methods) images are also used by the MedsApp to gather, train and use the data for autonomous medical procedure goals.

Quantum machine learning for medical procedures and applications runs at or about 14 qubit which runs at least 1,050 times faster than contemporary processing times of the MedsApp system without quantum technology.

Quantum key distribution is a secure communication method that implements a cryptographic protocol based on the laws of quantum mechanics that performs a secure method for exchanging encryption keys using the principles of quantum mechanics. MedsApp utilizes this system in some of its quantum subsystems.

In an embodiment of this disclosure, MedsApp utilizes quantum computing as a “MedsApp Training Coach” for autonomous robots, letting them explore millions of possible actions in parallel instead of learning by slow trial and error. The MedsApp hybrid quantum classical training module teaches robots safer, more precise motions more delicate, starting with more cumbersome and detailed physical medical procedures such head and neck surgeries. MedsApp can extend the same quantum platform across broader industrial and service robotics markets such as in the meat packaging, greenhouse evaluation, and janitorial applications.

This new disclosure utilizes classical and quantum algorithms for self-directed physical medical procedures and applications performed by robots, drones, and watercraft in the medical, dental, dermatology, cosmetic, ophthalmology, and veterinarian professions without the aid, intervention, or monitoring of human operators or clinicians. This new disclosure utilizes both classical and quantum computing algorithms to perform autonomous medical procedures that were either manually trained or self-trained to provide self-direction. The speed of the quantum computing algorithms enables this new disclosure to operate faster and more precise than classical algorithms. In every day regular conventional computers using classical software programs and classical algorithms, many testing, calculation, and evaluation procedures are one by one. In contrast, quantum computers (utilizing quantum computing) perform many types of calculations, while exploring many other possibilities at the same time that enable the system to find useful training patterns, and more precise procedures faster. MedsApp utilizes “quantum training” to train robots which in turn learns more precision in complex situations, like surgery, by allowing the quantum computer to search through huge numbers and datapoints of “what if I do this?” possibilities.

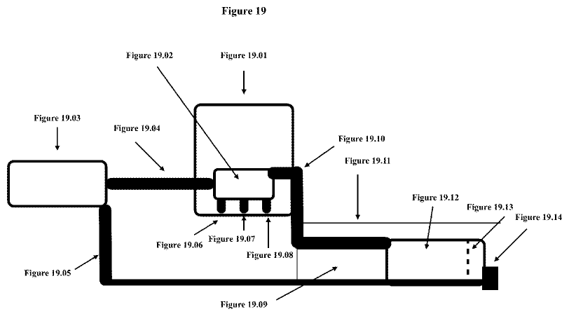

In an embodiment of this new disclosure, quantum computing technology is utilized to accelerate the processing of data, evaluation of data, training of data, and finally the autonomous self-directed medical procedures and applications without human being intervention, guidance, or monitoring. FIG. 19 is an overview of a quantum computer that is assisted with cooling by being placed underwater.

In an embodiment of this disclosure, mathematics both basic and advanced are programmed into algorithms. Mathematics and their corresponding calculating computer software programs equations are used to dot plot data, fill in unknown data and plot future medical procedure steps as well as forecast events using MedsApp data. The mathematical equations fill in the blank spaces with no known data that was not be obtained and or was skipped or the data was abnormal. Derivatives and associated equations are used to track the movements of training hands which can be doctors and or mechanical arms. The movements tracked from medical procedures by medical staff and mechanical fingers attached to mechanical arms can be derived using mathematics. The 3 separate medical procedural methods are used in combination to form a 3 dimensional view of a MedsApp operating room, a patient's body components (internal and external), and the MedsApp operating table which is further combined to form one 3 D view (over 3 dimensions when using quantum computing and quantum technologies) to archive the images, the markers, the measurements, and the positions in electronic form noting time and space. Mathematical dot plots (they fill in gaps in data that is unknown), mathematical imputation (fills in unknown data), mathematical derivatives (rate of change) mathematical outlier detection (data that falls outside of patterns), mathematical linear interpolation (unknown data values between two known data points) and basic mathematical equations are converted into computer software programs and algorithms to ascertain data that is known, unknown as well as dot plot data that is an outlier. Simulation software is utilized for training MedsApp using mathematics. MedsApp quantum computers, computer software devices, tablets and cell phones all use MedsApp simulation software. Simulation is used as a component to train the MedsApp and real time physical medical procedures. Mathematical simulation is also used for both training and physical autonomous medical procedures. For purpose of this disclosure, mathematics provides equations converted into simulation algorithms, 3 D images and video, and also matrixes and matrices (solving systems of equations).

In an aspect of this disclosure, MedsApp system's software and hardware, its methods and medical procedures can be combined at the onset of a self-directed physical medical procedure. In review of the autonomous 3 medical procedure methods, the following methods are: Twin Procedures which are based upon preprogrammed step by step instructions that are set before the procedure takes place, Bouncing of data in real time from the real time patient data and compared to pools of archived trained data from numerous prior patients pools of data, and finally Marked measured reference points which are measured distances from markers on a patient, markers on an operating table, and markers on patients body parts and organs for reference points with some points using 3 dimension areas in the x, y, and z axes (and k axes which continue in succession using quantum technologies) . The 3 methods: “Twin”(Twin Procedures), “Bouncing” (Bouncing back and forth of data), and “Markers” (Marked measured reference points) are called “the 3 MedsApp medical procedure methods”.

The following describes how the components listed in this specification work together to self-direct autonomous physical medical procedures without human operator intervention. As stated in this specification, when the MedsApp determines that a physical medical procedure should take place and an organ must be scanned, biopsied, or removed, a decision is made. With choices of three separate methods, the MedsApp decides on which one will be the leader with the other two on standby. Depending on patient data, the evaluation determines which method is chosen. In an aspect of this disclosure, if a method has been chosen for a physical medical procedure on a patient, the MedsApp at any time (before or at any time during the procedure) may loop back to another single method or both. As an example, the marker method was chosen for a medical procedure because the patient falls within a normal pool of age, weight, height and in good health with other data pool patients. After the procedure has started, it was discovered that the gallbladder was disintegrated (different than what was expected) , the bouncing method should be deployed since measurements and markers would not work, nor would the twin procedure since the data is skewed with a disintegrated gallbladder. In yet another example, if the measurement method is chosen and the patient has been in a car accident and body part are not where they should be, bouncing of data from prior patients with the same type of injury (broken ribs), as well bouncing of data of images would be the next choice. In yet another example, if the bouncing method is chosen first due to patient data that is limited in nature, after medical procedure starts, it was discovered that the patient had a hysterectomy which was not disclosed to the MedsApp ambient listening device when the MedsApp meet with the patient, the twin (archived data pools of digital twin of patients with hysterectomies was summoned by the MedsApp) and the twin method was deployed.

In certain aspects of this disclosure, two separate data pools must be propagated with a prodigious amount of patient data that are used for comparison with a high rate of comparison. Patient data includes but is not limited to patient images, patient measurements, and patient healthcare data. Both data pools consist of any and all types of patient data.

MedsApp technology foundation operates through self-direction. All of the listed sections of software and hardware in this specification contribute to certain training procedures or contribute to certain autonomous physical medical procedures. Both trainings and medical procedures also have many main foundational functions they both use. Of most importance are the two separate data pools, without either, the system will not work properly. One data pool is the real time (in the now) data obtained by the MedsApp system from the patient having a physical medical procedure being performed on. The second data pool is stored archived data on computer storage devices from numerous prior subjects obtained by the MedsApp system.

In an aspect of this disclosure, self-directed physical medical procedures rely on image comparison of two data pools. Those two pools of data being compared to each other are: image pool comparisons of patient data, measurement pool comparisons of patient data, movements and actions of physical procedures compared to patient procedures, and 3 dimensional views of all data between the two pools. When using quantum computing technologies, the dimension totals could be much higher.

In an aspect of this disclosure, each MedsApp system step compares images, measurements, actions, movements, and patient data. The step by step tasks ask three questions: 1—Is the real time data close or the same in prior training data? 2—Does the data obtained reach the threshold of the accuracy desired? 3—To what degree is are the differences? If there aren't any noticeable differences with the data being trained (data obtained from a new patient) compared to the data archived in the databases from prior patients, the system is instructed to move to the next steps. If there are noticeable differences between both sets of data pools, the algorithm is tasked to do 2 things. 1—adjust steps. 2—adjust actions. Then the system restarts processes with adjustments.

For purposes of this disclosure, tracking and mimicking are used both in training for self-directed procedures.

MedsApp mimicking through tracking measurement markers. In an aspect of this disclosure, MedsApp employs real time medical procedural movements by mimicking archived movements through prior medical procedures. Mimicking is employed for training data to be archived for future autonomous medical procedures. Mimicking is utilized in gloves, clothing, mechanical arms, mechanical devices and tools affixed to mechanical arms.

In an aspect of this disclosure, MedsApp mimicking is utilized in all 3 medical procedures methods. Tracking gloves with transmitting devices (MedsApp tracking and transmitting data devices) are used for mimicking (also called tracking) medical procedures in both archiving training data and autonomous medical procedures. Gloves, clothes (loose fitting and tight fitting) that can be powered by battery and be connected to a wire, (or use wireless transmissions through wireless transmission devices) is used by the MedsApp system for transmitting data. In an aspect of this disclosure, human operators (with fingers, hands, and wrists) press on (with glue and or adhesive) tracking devices or have the tracking devices embedded in gloves, headsets, and clothing for medical procedures that can be “mimicked “tracked” by wireless data transfer for training MedsApp systems.

In an embodiment of this disclosure, MedsApp utilizes many facets of tracking devices and tracking technologies tools for training as well as to perform physical medical procedures. Many of the motion tracking technologies use tracking and positioning markers on both the patient and the performing robot that is performing the physical medical procedure. In an embodiment of this new disclosure, medical tools, medical instruments, and medical devices are used with both training and autonomous medical procedures. The markers are affixed to the surfaces on the mechanical arms, mechanical hands, mechanical fingers, medical tools, medical instruments, and medical devices. The markers can be made of any material and be in any shapes with embedded transmission technology with some maintaining reflection ability. The transmission data technologies enable data to be transferred to MedsApp data storage devices for archival purposes. The data can also be used in real time. Markers can also be placed in the body and left there for data gathering before, during subsequent surgeries and post-surgery medical checkups. For purposes of this disclosure, measuring, mimicking, tracking, marking and positioning can relate to markers.

This new disclosure pinpoints organ areas through markers and is able to pinpoint an exact area of an organ, an exact placement of a medical tool or device for incision on patient, and further exact tool placement on and in a patients organ for removal by employing past imaging of patients organ positions (over 400,000 images) to present real time procedures on patients when removing an organ without human operator intervention. Reference point markers and devices are placed on and around MedsApp operating tables on which a patient is strapped onto for stillness with no movement whereby any physical medical procedure on a patient will transpire within the reference points. Mathematical equations fill in unknown data from patients when the procedure does not recognize related measurements of data pools affecting the next steps.

Baskets of data coded. MedsApp codes are formed that consist of baskets of data. The baskets of data include any and types of patient data. MedsApp codes can have two or more baskets of data. One such example can be a code with 5 different patient data types. An example is the code disGB150b509459 which may also have a corresponding condition, image with measurements and basic patient data. “disGB” stands for disintegrated gallbladder “150b” stands for weight (150 pounds) of patient “509” stands for 5 foot nine of which the corresponding image to the same code is a disintegrated gall bladder of a 150 lb patient that is 5 foot nine inches in height from a data pool of 459 patients with the exact same data points. The image or images may all be combined together to form one image with overlapping views when using quantum computing technologies. This technique is especially helpful with next self-directed steps when challenges arise in a self-directed physical procedure whereby next steps are limited.

In an embodiment of this disclosure, codes which are baskets of data are used to provide step by step instructions for performing a physical medical procedure autonomously.

For purposes of this disclosure, the physical medical procedures operate on a Cartesian Plane utilizing several axes. The x axis, y axis and z axis are called a 3 dimensional cartesian plane. When using the x, y, z, k, l, and d axes, quantum computing is used for the 6 dimensional levels and called the 6 dimensional cartesian plane.

In an embodiment of the new disclosure, communication of data is also in the form of direct communication between two or more robots, servers, cloud computing or from blockchain all known as two or more communication points in the MedsApp system. The back and forth communication between the robots can be in any form which includes but is not limited to conventional communication such as computer language, verbal, designated robot talk that is computer langue based and only understood by the MedsApp system or a combination of both. The transfer of data between the two communication points connects with every aspect of data from a real time patient data pool to archived patient data pools.

The application will work in a back and forth communication method with data between two connected entities for evaluation of data. The data can be converted from verbal to electronic data (or vice versa) in the form of measurements, images, and data of motion and action on an x axis, y axis, and z axis of a reference coordinate frame (3 dimensional cartesian plane) or refereed to reference frames axes in this specification and z axis with rotational data (roll, pitch and yaw). When using quantum computing there can be many superpositions and dimensions with an additional K, L, and D dimensions added on to the x, y and z. In an aspect of this disclosure, a robot will measure a distance with and without markers or position reference points and communicate that data to the MedsApp system for evaluation. The data evaluated can be evaluated in a different manner from other methods discussed in this disclosure. As opposed to other methods discussed in this specification such as measured lengths, image comparison in small increments of roll, pitch and yaw on the cartesian plane, this aspect only utilizes data in verbal form of talk between the robots and or machines. Two examples follow. The first is the distance between an area of a patients rib to 16 different positions of a patients stomach. This data is communicated not in mathematics but in coded references. In the archived database of areas from 16 different positions of a stomach to an area of a rib on many patients, that data was archived more than 51 times. The data was assigned a code of “LPLRCenterStm1throughSt8”—Lower part left rib bottom most center area of measurement to Stomach area 1, Stomach area 2, Stomach 3, Stomach area 4, Stomach area 5, Stomach 6, Stomach area 7, Stomach area 8. This code is used for evaluation to instruct the next set of instructions to the system using past measurements and data, from codes. These codes are in the millions and are used to produce the next steps for the MedsApp to self-direct medical procedures using codes without the interaction of human operators. The second example is a quantum technology example. If there are 17 superpositions with 32 cartesian plane dimensions, those corresponding codes will be in qubits and dictate not only a data point but attached and or embedded will be the next step in a long line of procedures. The code may be in the form a square block like that of a bar code or QR codes or just pictures that represent data. In another example of this disclosure, MedsApp has a developed a sensor triangular skin imaging system where exact points on skin, fur, or animal coats can be determined exactly by creating a high resolution map of an area of a skin in 2, 3, or 4 dimensions.

MedsApp production of 3 dimensional images of a patient utilizes many devices and tools listed in this specification, which include electromagnetic sensors and simulation software. This new disclosure produces 3 dimensional images utilizing trained data from the 3 medical procedure methods (Twin, Bouncing, Marker). Positioning (green screens may also be used) by measurement devices, positioning devices on and in a patient, a patient's body, a section of a patient's body, or a specific organ of a patient helps create 3D images of patient external and internal body parts. These 3D images are archived by MedsApp algorithms. The Twin method utilizes these images for bouncing data back and forth between real time client and archived patient data for comparison. In an aspect of this disclosure, 3 dimensional images of any and all components of a patient's body are created in real time and continually revised when more data is acquired from the patient.

In an aspect of this disclosure, Spacial Depth cameras and Calculations, ITOF devices (time of reflection), structured light devices, Dtof devices (time back and forth between sensors), LiDAR devices, and stereo vision devices are all used in the training and autonomous medical physical procedures being performed on patients. With computer software programs directing robots, the robots and sometimes drones can see things the way human operators do. To do this, depth sensing technology has evolved for machines. Machines using new technology can see things in 3-dimensional views. LIDAR in this new application utilizes 3D survey tracking. CAD models can also be utilized as well as shot plates. In an aspect of this disclosure, Spacial-3 Dimensional imaging is incorporated utilizing the x, y and z axis (and k if quantum computer is used) in medsapp.ai positioning of markers. In an aspect of this new disclosure, computer software programs with mathematical equations such as derivatives fill in areas of unknown patient data. An example would be that of a present real time procedure of a gall bladder removal with patient “Sarah” who weighs 85 pounds and is 87 years old. It is likely there is very little data in the prior patient archives of a 87 year old, having their gall bladder removed that weighs 85 pounds. If there is no data available for a past gall bladder procedure with this unique patient named Sarah, the MedsApp will employ mathematics. Dot plotting for purposes of this disclosure is defined as providing “data fills” for data that is unknown, maybe skewed or filling in between two data points that docent exist. In one example of Sarah, her gall bladder is small in size whereby data from prior patients archives will not provide the data fill. The data fill is the distance between Sarah's belly button (as a measurement point) and derive (in algorithmic code) where the next move in small increments in movements of the x, y, and z axis (3 dimensional) while at the same time utilizing another vantage point of Sarah's body (the bottom most position of her right rib) to compare distances from right lower rib edges of other larger younger patients. Upon a minimum of 12,200 data points which include images, distances, 3D archived images and video of Sarah, the medical procedure can start and 1, 2, or the 3rd medical procedure methods can be deployed. In an embodiment of this disclosure, looping feedback of numerous MedsApp algorithms is used for checking, double checking, and continual checking if the correct algorithms are being used. No human intervention is involved in any looping feedback, the process is self-directed by system operational hybrid algorithms that self-learn. All of a patients data from MedsApp will become patient data (archived patient data pools) used for comparisons to other patient data. A patient can be replaced by a mammal or flora that is s different type of patient. Patient data pools and all data points from humans, animals, plants, trees, and bugs are archived in various MedsApp databases. MedsApp server farms can be utilized for data storage.

In an aspect of new disclosure, robots performing physical actions during a procedure or application speak verbally of all their detailed movements and descriptions of actions for MedsApp ambient listening technology to train, store, evaluate, and compare the verbal data. The verbal data can be converted into measured data and vice versa. The data is used for training but also for autonomous physical medical procedures. The data is then combined with all physical data (movements tracked and or mimicked with tracking devices on robots). The data obtained can also be converted into a 3 dimensional map for training purposes. This is used if the system encounters challenges in performing a successful step by reviewing images, movements, verbal data, mimicked data for guidance. If during training or during an actual physical medical procedure, a challenge is encountered, the system will pause and view successful images, video, verbal information from robots, and tracking to exact moments and positions from many prior successful procedures. At this challenge point in the process, the system will slow down with data being evaluated from the present procedure to archived data for past procedures. Algorithmic loops will continue until challenge is resolved.

MedsApp Green screens are used to also track movements and create 3 dimensional views of a patient's body. Green screens are used in this new disclosure to track archive movements during the physical medical procedures which also use pen marcers which place dots (some indelible) on skin, organs and other internal body parts. The backgrounds must be green in order for the data mining. After utilizing the computer software programs, all data is exported to MedsApp databases. In an aspect of this disclosure, the data is utilized as the green screens being used in movie production for tracking actresses. Utilizing green screens in this new application to archive movements during the medical surgical procedure can use pen Tracking marcers (dots on skin, organs and other internal body parts). The backgrounds must be green as well as the operating table must be green. After utilizing the software, the data is exported to the MedsApp database.

In an embodiment of new disclosure, the entire system is autonomous, self-directed and performs medical procedures and applications without any human operator intervention. The machines, computer devices, robots, drones, and internal and external body water craft (also referred to as machines) utilize sensors, devices, tools and hybrid algorithms to make decisions and navigate their environments to perform simple tasks such as testing oxygen levels of a patient to complex tasks of such as organ removal. Reference points such as where oxygen testing devices should be placed on a patient by a robotic arm for testing (forehead) is trained in a repetitive manner whereby data is sometimes archived on over 400,000 placements of the oxygen testing device images and physical procedures. MedsApp robots can adapt to changing conditions and perform tasks in various settings, such as healthcare, assisted living facilities, and animal health care settings. The new disclosure has two types of training systems to perform the procedures and applications, one being manually trained system and the other being a self-training system.

MedsApp Motion tracking. MedsApp Motion tracking is the process of recording images and video of the spacial alteration of the movement of a human operator or a thing. The spacial change can be movement between the x, axis, y axis, and z axis as well as change in velocity. Video games utilize motion tracking to animate and simulate objects which is referred to as computer generated imagery or “CGI”. In an aspect of this application, biomechanics is used which is the study of movement of people, animals and insects. The metrics, patterns and movements of medical staff and or robots performing the procedures are converted into electronic form and stored in MedsApp databases for training as well as archival of data for self-directed physical medical procedures. MedsApp CGI, is the use of computer graphics to create still or animated visual content for movies, video games, and other media. It is used for both training and self-directed procedures. It also allows for the creation of realistic images and special effects that enhance storytelling and visual experiences. This application is also used to explain what the patient may go through during a procedure and or application with the patient's head and body superimposed into the computer graphics.

MedsApp.AI CGI. In an embodiment of this new disclosure of this new disclosure, CGI will be used for tracking and tracing movements.

Camera and Video Mimicking. In an embodiment of this new disclosure of this new disclosure, cameras and video are used for tracking and to mimic physical movements.

MedsApp.AI Sensors may include 3D optical imaging (stereo vision, structured light, or LiDAR), force and torque sensors, acoustic or ultrasonic scanners, thermal sensors for biological monitoring, physiological sensors (ECG, pulse oximetry) depending on procedure. MedsApp data from sensors are fused with other obtained data of the patient to produce an anatomical map, instrument tissue interaction profile, and procedural state assessment. They are used for both training and self-directed procedures.

Procedural Intelligence Module. The MedsApp module uses machine learning models trained on procedure and specific datasets, broad datasets, and data pools. The MedsApp functions include predicting next procedural steps, recognizing anatomical structures, features, and boundaries, monitoring tool trajectory and tissue interaction, identifying abnormal events or deviations from expected patterns.

Self-Directed Control Algorithms. The MedsApp algorithms generate motion trajectories optimized for safety, precision, and efficiency. The capabilities include real time path planning with constant false loop feedback, adaptive force control during tissue manipulation, autonomous micro adjustments based on feedback vectors, predictive modeling of anatomical movement or deformation.

Safety and Intervention MedsApp Module. The system continuously assesses operational risk by evaluating: Force thresholds, anatomical proximity alerts, physiological sensor data, and deviations from expected procedural states. When thresholds are exceeded, the system may halt autonomously, revert to safe pose, or may request human operator control.

Robotic Manipulator and End Effectors. The robotic manipulator includes multiple degrees of freedom, precision actuators, and interchangeable tools designed for: medical procedures (e.g., incision, suturing, ablation), dental procedures (e.g., drilling, scaling, polishing), and veterinary procedures (analogous to medical procedures with species specific considerations). MedsApp-guided intraoperative monitoring has advanced the robotic surgical technologies by providing real time decision making algorithms and predictive analytics “forecasting events as well as next steps”. MedsApp has employed comparative image-based tissue samples (normal tissues compared to abnormal tissue samples), guiding the MedsApp to successful surgical procedures by using the comparative image MedsApp component to guide further steps in the procedure such as does the tissue show cancerous “abnormal” cells.

Medical tools and medical devices with tracking objects explained. The MedsApp medical tools, medical instruments, and medical devices that are used with the MedsApp training applications utilize tracking bright colored objects that are affixed to the surfaces on the medical tools, medical instruments, and medical devices. The objects can be made of any material and be in any shapes. The objects can also be covering a part of or replace a section of the medical tools, medical instruments, and medical devices for tracking purposes. The objects may also be reflective. Positioning of Tracking Markers can also be placed in the body for data gathering before, during subsequent surgeries and post surgery medical checkups.

MedsApp.AI LiDAR sensor devices. MedsApp LIDAR or Light Detection and Ranging use a focused laser emitter that moves back and forth to scan and project a pattern of points of light within the area it is recording. It is used for both training and self-directed procedures. Each time a pulse of light (or reflection) is projected from the LIDAR to an area on the patient and projected on the hands of the medical professional (including fingers and the medical instruments and medical devices) and projected on the mechanical hands (including fingers and the medical instruments and medical devices) the following happens. Sensor devices records the duration of time the pulse of light is emitted and the time it is reflected back to the sensors. During certain medical surgical procedures such as a knee replacement, mirrors can also be used to train new technologies. MedsApp LiDAR sensor devices utilizes quantum technologies as well as quantum computing.

MedsApp LIDAR scanners. MedsApp LIDAR scanners utilizes red lasers that can also produce single points of data such as where a mole is located on patient. It is used for both training and self-directed procedures. These types of data points can be as low as one up to many thousands used for measurement of distances. This process is called point cloud to pinpoint one mole. A 3D map can also be constructed using these point clouds. MedsApp LIDAR scanner devices and red lasers utilizes quantum technologies as well as quantum computing.

MedsApp Lidar scans. LIDAR in this new disclosure utilizes 3D survey tracking. CAD (computer aided design software coded and or revised for MedsApp) models can also be utilized as well as shot plates (two or more separate images). MedsApp LIDAR (light detection and ranging) scanner devices utilizes quantum technologies as well as quantum computing. It is used for both training and self-directed procedures. Red lasers are also used by MedsApp robots to point to certain areas of interest when training.

This new disclosure has been trained for medical surgical procedures, but the same procedures, methods, applications and operations can be applied to other types of industries. One such industry is a slaughterhouse, retail pet industries as well as in military combat applications.

MedsApp Digital scent technology. MedsApp Digital scent technology is both a mechanical and an electronic device that detects smells and scents in a surgical operating room to gather data in the form of categories and subcategories of odors or odours to be archived in a database.

MedsApp Lasers. The MedsApp lasers are used for measuring areas in and on a subject, the MedsApp operating rooms, tables, medical professionals, robots, machines, and the surrounding data such as lighting, and number of machines/medical professionals, type and number of surgical utensils utilized, and ambient temperatures. They are also used on subjects during medical surgical procedures. The MedsApp lasers can also utilize quantum technologies as well as quantum computing. The MedsApp utilizes lasers that are used to track the physical medical surgical procedures by human operators whereby the software used in computer software programs is used in printer scanner devices. Specific code can also be used to form different computer software programs to scan in a higher resolution. The specific code can also be used to obtain video of the physical medical procedures.

MedsApp cameras collect all movements by both the machines and the human operators when training before, during and after the physical medical procedures. The cameras recording all movements on a 3 dimensional view as in the x axis, y axis and the z axis. In an aspect of this disclosure, more than one camera is used whereby there can be over 100 cameras. The MedsApp cameras can also utilize quantum technologies as well as quantum computing.

MedsApp machines include robots with mechanical arms, drones with mechanical arms of which both can retrieve tools from a surgical device tool box, replace, or put back the tools (medical surgical devices and tools) into the surgical tool box, utilize medical surgical devices to perform medical procedures, clean the medical surgical devices, repair the medical surgical devices in real time or perform any other action has made in an operating room or will be made in a surgical operation room or medical application. The MedsApp robots and drones operate a 3D printer in the MedsApp operating room to construct real time body parts for a patient or broken parts of a medical device if needed.

MedsApp Multi Sensor Arrays. The multi-modal sensor arrays are also in used in MedsApp googles which can be affixed to mechanical arms, mobile vehicles and can be combined such as cameras plus LiDAR plus radar. AR/VR MedsApp headsets (also called MedsApp googles) for inside-out tracking, mixed reality surgical purposes. MedsApp body surveillance systems for abnormal anything including movement, behavior, texture, color, form, or heat transfer such as an untethered small drone as to not disturb the procedure setting, the drone can also be utilized to speak the results to the doctor from the results from the UMMDA for training purposes. The MedsApp array feeds data into the MedsApp algorithms (SLAM-Simultaneous Localization Mapping), sensor fusion, and tracking devices to produce data such as 3D point clouds (set of data points in space) or meshes of the surroundings, accurate pose (where is the MedsApp as in 3D space?), object detection and tracking in 3D, lighting intensity devices “candle power” automation autonomously by machines, nanotechnology devices that are miniature in dimensions and weight (nanometers and nano grams).

MedsApp 3D spatial perception. In an embodiment of this disclosure, MedsApp 3D spatial perception builds a live, internal model of the shape of the body (walls of body component and asks itself-is it normal? are there obstacles/obstructions?). The position and motion of body component, such as the heart and is the heart pumping normal? The system's own position and orientation (localization) as to the patient is also learned in an expedited manner.

MedsApp utilizes communication connections such as wired, wireless, satellite, laser, quantum technologies, quantum-enhanced lasers, via light sources such as filter optics, or an optic light language such as morse code, or quantum key distribution. A combination of the prior communication forms is also used by MedsApp. MedsApp hardware components can also be control units (both software and hardware) for mobile vehicles with and without mechanical arms, mobile vehicles for land, air, and water, medical tools, stationary mechanical arms, medical devices, tools, mechanical, electronic devices, optical devices, image enlargement devices, LiDAR devices, pressure devices, thermal devices, spacial devices, imaging sources (CBCT, MRI, ultrasound), embedded processing devices, multi-modal sensor array devices for 3D spatial perception, physiological monitoring devices (physiological monitoring systems are devices designed to measure and track vital signs and other health parameters in patients, often using non-invasive methods), contact force detection devices, procedural intelligence module trained on patient specific models to predict procedural steps, identify anatomical structures, and monitor state progression, control MedsApp algorithms capable of generating, modifying, and validating robotic tool trajectories in real time.

The multi-modal sensor array for 3D spatial perceptions utilizes different types of sensors working together allowing the system to understand the 3D world around it such as a patients body and its components.

The new disclosure discussed in this specification is a technological breakthrough. Present public opinion is that “medical artificial Intelligence” or better known as algorithmic code that enables a machine to act autonomously cannot perform physical medical procedures without a human doctor. The consensus among the general public is that a physical medical procedure needs the training of a doctor, their experience in the operating room, and their quick thinking decision making abilities that a machine cannot do. In this specification, the impossible has been done. Physical medical self-directed procedures by machines utilizing trained data have completed autonomous medical procedures without intervention, guidance and decision making from doctors and human operators.

There are numerous methods that enable and assist the MedsApp algorithms to successfully complete a medical procedure with the foundation of the technology being that of trained data from prior patients used to complete a real time “in the now” medical procedure of a patient. Each and every time a successful or unsuccessful physical medical procedure in real time is completed, that data becomes prior patient data and is archived in a MedsApp.ai storage device for future use.
The first step in any MedsApp procedure in the intake of a patient which is the intake of patient data in combination of the new patient's historical data. All returning patients do maintain new data, even if the return is two days apart from the last visit.
MedsApp performs both self-directed physical medical procedures (invasive) and physical medical applications (non-invasive). Invasive being biopsies and organ removals and the like while non-invasive being automated oxygen testing levels of a patient by robots and automated sputum testing. The entire intake of a patient is automated until the patient is excused and released by the MedsApp. During visits, the system updates data germane to the patient such as cancer rates in the hometown the patient was born in.
The new algorithmic “artificial intelligence” technology “MedsApp.ai” is a training system of data and then the implementation of that trained data which performs autonomous physical medical procedures and applications without human operator intervention. The new algorithmic technology system deploys quantum technologies, quantum computing, and classical algorithms to manually train and self-train data from every patient while creating new written computer software language code (both python and qubits) that also trains data as well as perfects self-directed physical medical procedures.
The new MedsApp technology utilizes combinations of methods, hardware, and software components. The components detect body parts and areas of a patient with imaging, video, measurements and positions of body parts from markers which form 3 dimensional views (cartesian x, y, and z axes) of those patient body areas (6 dimensional views are used with quantum superpositions) whereby that data is archived in numerous different ways and types for archival purposes. The data could have embedded marker points on images of patients which are then measured to markers on surgical operating tables as well as the Cartesian plane to obtain exact positions of body parts and areas. There are two data pools that the system maintains, one pool of data is in real time (in the now) of present patient data. The second is the trained data from numerous prior patients data that is archived that the first pool is compared to. The first pool is compared to the second pool of data for step by step instructions to perform a physical medical procedure. The system remains in the constant “bouncing” stage of back and forth data between the real time procedure to archived data for self-direction instruction. The data is compared in a prodigious manner whereby a single image and movement of a medical tool one centimeter may contain billions of 4 D views, images, measurements and markers. The data pools bounce similar patient data between the real time data to archived data such as a health score of a patient (good, bad and average scores with bad scores relating to prior health issues of a patient such as cancer). Health scores, age of patient, height, weight, prior health history and pre-birth data such as exposure to the Flint, Michigan water crisis, assists with measurements of body organ (maybe that the liver, stomach, or kidneys of the patient was affected by tainted water consumption) such as colors, abnormal images, and measurements of an organ (bile duct to what was supposed to be a portion of a gallbladder) to the lower right third rib that are outliers, forecasted health issues and present health related data such as smoking cigarettes.
With enough training data for specific physical medical procedures, the performing of such a procedure is self-directed. Some training may take years to complete such as open heart surgeries that maintains enough training for the MedsApp self-directed system to give the green light for an automated step by step procedure of a specific physical medical procedure.
The described embodiments are illustrative and are not intended to be limiting. Variations and combinations of the disclosed features may be implemented without departing from the spirit and scope of the invention as defined by the claims. It is the inventors wish that this technology saves lives of people, plants and animals. Month of March in the year 2026.

1. A self-directed robotic system for autonomously performing a physical medical procedure on a subject, comprising:
(a) a training subsystem configured to obtain and store training data in an archived data pool and to train or select one or more models;
(b) a procedure-execution subsystem including a robotic apparatus having at least one actuator configured to move at least one instrument relative to the subject and one or more sensors configured to generate real-time procedural data during the physical medical procedure;
(c) a real-time data pool associated with the subject; and
(d) a controller comprising at least one processor and memory storing instructions that, when executed, cause the controller to:
(i) access the archived data pool and the real-time data pool;
(ii) determine a procedure plan based at least in part on comparisons between the real-time procedural data and the training data in the archived data pool; and
(iii) execute the procedure plan by autonomously generating and issuing control commands to the robotic apparatus, wherein the physical medical procedure is executed without real-time guidance or intervention by a human operator.

2. An autonomous physical medical procedure system determining the procedure plan comprises repeatedly comparing the real-time procedural data to the training data in the archived data pool at a selected time granularity to select subsequent actions during the physical medical procedure, comprising:
(a) executing the procedure plan comprises validating one or more planned steps at checkpoints and autonomously updating the procedure plan when observed real-time procedural data deviates from an expected procedural state;
(b) the controller computes a difference metric between the real-time data pool and the archived data pool and selects at least one control action to reduce a deviation from an expected procedural state;
(c) the controller stores a procedure state at each checkpoint and, responsive to detecting the deviation, autonomously rolls back to a stored procedure state or branches to an alternate action sequence;
(d) wherein the controller groups features associated with a procedure step into indexed data structures stored in the archived data pool and retrievable as step definitions during execution of the procedure plan;
(e) wherein determining the procedure plan comprises selecting or generating a step-by-step plan prior to initiating the physical procedure based on similarity between the real-time data pool and the archived data pool.

3. A method of autonomously performing a physical procedure on a subject, comprising:
(a) generating, by one or more sensors, real-time procedural data during the physical procedure;
(b) accessing an archived data pool storing training data from prior subjects or prior procedures;
(c) determining, by a controller, a procedure plan based at least in part on comparisons between the real-time procedural data and the training data in the archived data pool; and
(d) autonomously controlling a robotic apparatus to execute the procedure plan by issuing control commands that move an instrument relative to the subject, wherein the physical procedure is executed without real-time guidance or intervention by a human operator.

4. The system of claim 1, wherein the controller is configured to generate, update, or select trained model parameters using training data compiled by at least one of (i) human operator performed demonstrations and (ii) self-training by the system, and to utilize the trained model parameters to execute the autonomous physical medical procedure wherein the controller represents at least one of the subject, the instrument, or the robotic apparatus in a coordinate system including x, y, and z axes and further tracks one or more orientation degrees of freedom.

5. The system of claim 1, wherein the training data comprises (i) a first data pool including real-time data acquired during procedures performed on one or more patients and (ii) a second data pool including archived data from a plurality of prior subjects.

6. The system of claim 5, wherein executing the procedure plan includes at least one comprising:
(a) comparing images or videos in the first data pool to images or videos in the second data pool;
(b) comparing measured values in the first data pool to corresponding measured values in the second data pool;
(c) comparing a sequence of procedural steps in the first data pool to a reference sequence in the second data pool;
(d) comparing data obtained from UMMDA in the first data pool to a reference sequence in the second data pool.

7. The system of claim 5, wherein the controller executes software routines implementing at least one of: trigonometric functions, ratios and proportions, interpolation, extrapolation, regression, averages or weighted averages, constraint and logic evaluation, probability or statistical inference, imputation, derivatives, outlier detection, and linear interpolation, to (i) validate known data, (ii) infer unknown data, and (iii) flag outlier data.

8. The system of claim 1, further comprising one or more markers positioned on the subject or in an environment of the subject, an operating surface, the instrument, or the robotic apparatus, wherein the controller uses the one or more markers as reference points for positioning or navigation which determines at least one of position, orientation, or trajectory constraints based on measured distances among the one or more markers wherein the controller triangulates marker positions in a Cartesian reference frame and validates an instrument pose using the triangulated marker positions.

9. The system of claim 1, wherein the controller comprises at least one of a CPU, a GPU, a tensor processing unit (TPU), a neural processing unit (NPU), a field programmable gate array (FPGA), and an application-specific integrated circuit (ASIC) accelerator, a DSP, an FPGA, a microcontroller, or a system-on-chip device.

10. The system of claim 1, further comprising a quantum computing device including qubits, wherein the controller executes at least one quantum-assisted computation in generating at least one of the comparisons between the real-time procedural data and the training data, optimization of the procedure plan, or training of the one or more models.

11. The system of claim 1, wherein the system is configured to automate patient intake by acquiring and evaluating vital signs using one or more sensors, hybrid algorithms and UMMDA.

12. The system of claim 1, wherein the medical instrument comprises at least one of a tool, device, or machine component used in performing the self-directed physical medical procedure.

13. The method of claim 1, wherein determining the procedure plan comprises using training data compiled through at least one of (i) human-performed demonstrations and (ii) self-training by the system.

14. The system of claim 3, further comprising one or more tracking devices configured to track at least one of subject anatomy, instrument pose, or robotic apparatus pose, wherein tracked data is stored as archived data pool for training data and for future procedures.

15. The system of claim 1, wherein the training data and the real-time procedural data comprise multi-modal data including at least two of images, video, depth data, point clouds, ultrasound data, CBCT data, MRI data, force data, torque data, tactile data, physiological signals, instrument pose data, trajectory data, and data from a matter detection apparatus utilizing quantum computing technologies with qubits configured to test a matter sample from the subject and output test results to the controller for use in computing the procedure demand score or determining the procedure plan.

16. The system of claim 1, wherein the controller autonomously directs operation of one or more machines, devices, or tools used to perform the autonomous physical medical procedure either an invasive procedure or a non-invasive procedure in at least one of medical, dental, cosmetic, dermatology, ophthalmology, or veterinary domains.

17. The system of claim 1, wherein the controller processes stored data to forecast one or more future events, and wherein the system is configured for use in at least one profession other than medical, dental, cosmetic, dermatology, ophthalmology, or veterinary domains.

18. The system of claim 1 that utilizes an autonomous untethered roaming robot placed into a subject using at least one onboard sensor of the roaming robot.

19. The system of claim 1, wherein the controller implements a safety module that, responsive to a challenge condition, autonomously transitions the robotic apparatus to a safe state and triggers escalation to a human operator when the challenge condition is not rectified after (i) 400,000 evaluation loops or (ii) 7 minutes, whichever occurs first.

20. The system of claim 1, wherein the robotic apparatus and the controller are disposed in a mobile platform configured to travel to a location and perform the physical medical procedure in the mobile platform wherein the mobile platform further comprising a three-dimensional printer controlled by the controller, wherein the controller causes the three-dimensional printer to fabricate an artificial body part based on real-time measurements and to update fabrication parameters based on real-time measurement comparisons.
